(19) United States
(12) Patent Application Publication
(10) Pub. No.: US 2026/0033942 A1
(43) Pub. Date: Feb. 5, 2026

(54) Ophthalmic Devices, Systems and/or Methods for Management of Ocular Conditions

(71) Applicant: Brien Holden Vision Institute Limited, Sydney, NSW (AU)

(72) Inventors: Hassan Esfandiarijahromi, Sydney, NSW (AU); Arthur Back, Sydney, NSW (AU)

(21) Appl. No.: 18/994,429

(22) PCT Filed: Jul. 14, 2023
(86) PCT No.: PCT/IB2023/057223

§ 371 Date: Jan. 14, 2025

Related U.S. Application Data
(60) Provisional application No. 63/389,503, filed on Jul. 15, 2022.

(51) Int. Cl. A61F 2/16; G02C 7/02; G02C 7/06

(52) U.S. Cl. CPC A61F 2/1602; A61F 2/1613; G02C 7/022; G02C 7/06

(57) ABSTRACT

An ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile.

### CROSS REFERENCE TO RELATED APPLICATIONS

This application claims priority to U.S. Provisional Application No. 63/389,503, filed Jul. 15, 2022. This priority application is herein incorporated by reference in its entirety.

This application is related to International Application No. PCT/AU2013/00354 filed Apr. 5, 2013, International Application No. PCT/AU2013/001137 filed Oct. 4, 2013, International Application No. PCT/IB2020/057863 filed Aug. 21, 2020, International Application No. PCT/IB2021/057720 filed Aug. 23, 2021, International Application No. PCT/IL2005/000868 filed Aug. 11, 2005, International Application No. PCT/IL2011/000140 filed Feb. 9, 2011, International Application No. PCT/IL2011/000141 filed Feb. 9, 2011, International Application No. PCT/IL2011/000142 filed Feb. 9, 2011, International Application No. PCT/IL2011/000143 filed Feb. 9, 2011, International Application No. PCT/IL2011/050069 filed Dec. 11, 2011, and International Application No. PCT/IL2012/050538 filed Dec. 20, 2012. Each of these related applications are herein incorporated by reference in their entirety.

### TECHNICAL FIELD

This disclosure relates to ophthalmic devices, systems and/or methods for correcting and/or treating refractive errors and/or conditions of the eye. More particularly, this disclosure is related to ophthalmic devices, systems, and/or methods for correcting and/or treating refractive errors and/or conditions of the eye using an ophthalmic lens with one or more pulses configured to control an optical wavefront.

### BACKGROUND

The discussion of the background in this disclosure is included to explain the context of the disclosed embodiments. This is not to be taken as an admission that the material referred to was published, known, or part of the common general knowledge at the priority date of the embodiments and claims presented in this disclosure.

Ophthalmic devices incorporating simultaneous vision and/or extended depth of field optics may be used for presbyopia correction or for treating refractive errors including myopia control. However, there is a need for improved efficacy and/or flexibility with use of such devices.

Accordingly, there is a need to improve the performance of and/or increasing the flexibility of design associated with ophthalmic devices. The present disclosure is directed to solving these and other problems disclosed herein. The present disclosure is also directed to pointing out one or more advantages to using exemplary ophthalmic devices, systems, and methods described herein.

### SUMMARY

The present disclosure is directed to overcoming and/or ameliorating one or more of the problems described herein.

The present disclosure is directed, at least in part, to ophthalmic devices and/or methods substantially as described herein.

The present disclosure is directed, at least in part, to ophthalmic devices and/or methods for correcting, slowing, reducing, and/or controlling the progression of myopia.

The present disclosure is directed, at least in part, to ophthalmic devices and/or methods for correcting or substantially correcting presbyopia.

The present disclosure is directed, at least in part, to ophthalmic devices, systems and/or methods to correct and/or treat refractive errors and conditions of the eye including e.g., presbyopia, myopia, astigmatism, binocular vision disorders and/or visual fatigue syndrome.

The present disclosure is directed, at least in part, to an ophthalmic lens configured to correct and/or treat at least one condition of the eye (e.g., presbyopia, myopia, hyperopia, astigmatism, binocular vision disorders and/or visual fatigue syndrome) comprising: a base power profile; and at least one pulse selected to modify the base power profile.

The present disclosure is directed, at least in part, to an ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile, wherein the at least one pulse is selected to create a power profile with much greater precision than possible with other lens design techniques by using the one or more pulses to create an optical design configured to provide a higher resolution specification of a power profile.

The present disclosure is directed, at least in part, to an ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile, wherein the one or more pulses may be introduced to a base power profile to modify (e.g., increase, decrease, eliminate or create) one or more RIQ values (e.g., peak RIQ values) at any desired vergence along an RIQ curve. For example, in some embodiments, a bifocal base power profile may have two RIQ peaks—one at a distance vergence and one at a near vergence and one or more pulses may be introduced that create a new RIQ peak at an intermediate vergence to create a trifocal ophthalmic lens. In some embodiments, this design may also target the redistribution of a defined light energy away from the two bifocal RIQ peaks and/or a plurality of pulses may be designed to introduce multiple spaced apart RIQ peaks or a broader continuous RIQ region between the distance and near RIQ peaks of the base power to create an extended depth of focus (EDOF) while modifying (e.g. reducing) the light energy directed to the two bifocal peaks formed by the conventional pulses.

The present disclosure is directed, at least in part, to an ophthalmic lens configured to correct and/or treat at least one condition of the eye (e.g., presbyopia, myopia, hyperopia, astigmatism, binocular vision disorders and/or visual fatigue syndrome) comprising: a base power profile; and at least one pulse selected to modify the base power profile, wherein the one or more pulses may be introduced to a base power profile (e.g. a multifocal base power profile formed without pulses) based on the simultaneous vision principle and the one or more pulses may be configured to selectively modify an RIQ at any vergence (e.g. increase, decrease, eliminate and/or create peaks, across the useful range of vergences for vision). In some embodiments, the one or more pulses may reduce the light energy of the RIQ plot at the selected vergences to a relatively low level while maintaining it sufficiently high for good vision at other vergences (e.g., distance, intermediate, near and/or anywhere in between). In some embodiments, the one or more pulses may be configured so that the undesired light energy is spread across a range of vergences (e.g., outside the useful range of vergences for presbyopic vision). In some embodiments, the one or more pulses may be configured to modify interference from a first light energy on a first image plane (e.g., increase, reduce, substantially reduce and/or minimize the interference). In some embodiments, the light energy may be widely spread and of relatively low energy at the vergence (e.g., a low RIQ) including within the useful range of vergences.

The present disclosure is directed, at least in part, to an ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse (e.g., a plurality of pulses) selected to modify the base power profile and to form one or more on-axis focal points in front of, on, and/or behind a retinal image plane and/or control a focal point energy level at one or more image planes (e.g., modify, eliminate, reduce, and/or increase a focal point energy at a particular image plane, including reducing a peak focal point energy).

In some embodiments, the ophthalmic lens may be configured to modify a distribution of light energy across an image plane. For example, the light rays forming the spot diagram at an image plane may be redistributed (e.g., more uniformly spread and/or reducing light ray gaps in the spot diagram or plateauing in the enclosed energy profile (e.g., outside the centroid)) to improve night vision, night vision driving and alleviate night light disturbances.

The present disclosure is directed, at least in part, to a method for changing an image quality of an ophthalmic lens design comprising: identifying a base power profile of the ophthalmic lens design; obtaining an image quality metric (e.g., an RIQ) of the base power profile of the ophthalmic lens design; and adding one or more pulses to the base power profile of the ophthalmic lens design to generate a modified power profile that changes the image quality metric (e.g., reduces the RIQ and/or reduces a peak RIQ) of the ophthalmic lens design. In some embodiments, the one or more pulses may modify the RIQ at one or more targeted vergences. In some embodiments, the modification may be within the visually usable range of vergences used for vision (e.g., +/−3D). In some embodiments, the modification may be beyond the visually usable range to spread unwanted or unnecessary or not needed light energy (e.g., in an on-axis and/or off axis direction). In some embodiments, the modification may modify the interference of out of focus light energy on the light energy of at least one focal plane within the useful range of vergences (e.g., along a depth of focus or at any other selected vergence or at off-axis image planes). In some embodiments, the one or more pulses may provide an improved optical design tool to redistribute (e.g., precisely redistribute) light energy to or away from or across an image plane. In some embodiments, the one or more pulses may enable the optical characteristics of an ophthalmic lens to be specified with greater (e.g., higher) resolution so that light energy from an ophthalmic lens may be also directed at a higher resolution to modify the retinal image quality and/or light energy at each focal point with greater precision. In some embodiments, the one or more pulses may be designed not to alter the RIQ but may be used to modify (e.g. introduce or eliminate or increase or decrease) an optical artefact or phenomenon that may be desirable or not desirable for a wearer of the ophthalmic lens (e.g. a light disturbance effect, a halo or a starburst, a ghost image, a dysphotopsia diffraction effect from a junction, design configuration or shape (e.g. IOLs positive and/or negative dysphotopsia) or a prismatic effect, a chromatic effect, a lens distortion, and/or an image jump or image swim). In some embodiments, the artefact may be a residual from a manufacturing process or an inherent design feature of an ophthalmic lens that may not be readily modifiable. In some embodiments, the artefact may be an interaction of the wearer ocular characteristics and a lens shape or design configuration (e.g., a pupil size or an ocular aberration and optical zone size or an intraocular lens shape and positioning within the anterior or posterior chamber).

The present disclosure is directed, at least in part, to an ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse (e.g., a plurality of pulses) selected to modify the base power profile and to form one or more on-axis focal points in front of, on, behind and/or across a retinal image plane; wherein the ophthalmic lens is configured to provide a depth of focus that provides continuous (e.g., substantially continuous) vision from an object at distance to an object in the near vergences while focusing on a distance object.

In some embodiments the depth of focus may provide a partial depth of focus at near objects and/or distance object and/or intermediate objects.

In some embodiments the at least one pulse may be configured to create a diffraction effect to produce an image inside the human eye.

In some embodiments the at least one pulse is located on a front surface and/or a back surface of the ophthalmic lens (e.g., within an optical zone or outside an optical zone).

In some embodiments, the at least one pulse may be formed in the matrix of the ophthalmic lens (e.g., between the front and back surfaces).

In some embodiments, the at least one pulse may be formed on a layer, a film, a lens precursor (e.g., a mold tool), a casting mold, a lens blank and/or formed on a fully finished lens as a post processing step.

In some embodiments, the at least one pulse may be formed by a material additive process (e.g., printing, stamping, dying, 3D printing, and/or a lithographic process).

In some embodiments, the at least one pulse may be formed by a material subtractive process (e.g., lasers, light energy, CO2 or UV, etching, thermal, and/or a chemical process).

In some embodiments, the at least one pulse may be formed by a material modification process (e.g., thermal, chemical changing the shape, refractive index and/or a chemical composition modification process).

In some embodiments the at least one pulse has a width of less than 0.3 mm, less than 0.5 mm, and/or less than 0.75 mm.

In some embodiments the at least one pulse has a width of about 0.05 mm.

In some embodiments the one or more pulses comprises 1, 2, 3, 4, 5, 6, 7, 8, 9, 10, 11, 12, 13, 14, 15, 20, 25, 30, 35, 40, 45, 50, 60, 70, 80, 90, and/or 100 pulses.

In some embodiments the one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).

In some embodiments the at least one pulse has a height (e.g., a dioptic power (D)) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).

In some embodiments the at least one pulse has a shape selected from any combination of one or more of a square, conjoined of different features, triangles, rounded top, curved tops, slanted, oblique sides, and/or transition on the sides. In some embodiments, the sides of a pulse may be parallel to each other (e.g., substantially parallel) or not parallel to each other. In some embodiments, the tops of the pulses may be at least one of a planar, not planar, curved, monotonic, non-monotonic, periodic, and/or aperiodic profile (e.g., power profile).

In some embodiments the at least one pulse is a rapid, narrowband change in power.

In some embodiments the at least one pulse comprises a change in power comprising an increasing power component, a peak power component, and a decreasing power component relative to the base power profile. In some embodiments, the sides of one of the at least one pulse may be connected to the base surface and a peak (or top) of the pulse may not be connected to any other feature on the lens.

In some embodiments a first derivative of the pulse optical/power profile is discontinuous at a boundary between the base power profile and the at least one pulse. In some embodiments, the at least one pulse may include a discontinuity in the first derivative of the power profile in a least one location of the at least one pulse.

In some embodiments the pulse is a narrow width feature having a predefined width, power and/or position selected to change (e.g., reduce) at least one of the variability and magnitude of an RIQ associated with the base power profile. In some embodiments, the change in RIQ may be at any portion of the RIQ plot. In some embodiments, the change in RIQ may be at a particular vergence.

In some embodiments one or more of the at least one pulses is configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%.

In some embodiments the at least one pulse is a positive powered pulse relative to the base power (e.g., the base power of the lens at the start of the pulse).

In some embodiments the at least one pulse is a negative powered pulse relative to the base power (e.g., the base power of the lens at the start of the pulse).

In some embodiments, the at least one pulse (e.g., a relatively more positive, and/or negative pulse than the base power profile) may be configured to influence an extended depth of focus (EDOF). In some embodiments, a relatively more positive powered pulse may be configured to influence (e.g., change) the RIQ at any vergence depending on its height, width, shape, tilt, and/or location (e.g., a positive pulse may influence the RIQ over a distance vergence or an intermediate vergence or a near vision vergence or an EDOF vergence range or beyond the visually useful range of vergences from far to near). In some embodiments, a relatively negative powered pulse may be configured to influence (e.g., change) the RIQ at any vergence depending on its height, width, shape, tilt, and/or location (e.g., a positive or negative pulse may influence the RIQ over a distance vergence or an intermediate vergence or a near vision vergence or an EDOF vergence range or beyond the visually useful range of vergences from far to near).

In some embodiments the at least one pulse is a relatively positive powered pulse and a relatively negative powered pulse.

In some embodiments the ophthalmic lens comprises a substantially equal number (e.g., 1, 2, 3, 4, 5, 6, 7, 8, 9, or 10) of relatively positive powered pulses and a relatively negative powered pulses.

In some embodiments the one or more pulses may have a power to correct or contribute to one or more of a distance, intermediate and/or a near power (e.g., the one or more pulses may provide a depth of focus). In some embodiments, the at least one pulse may have a power configured for the treatment of at least one refractive error (e.g., a myopic defocus and/or a hyperopic defocus) and/or may contribute (e.g., enhance or diminish) an optical signal or retinal image quality associated with a vision correction or vision treatment or ocular condition.

In some embodiments the one or more pulses do not correspond to one or more of a distance, intermediate and/or near power.

In some embodiments the one or more pulses are conjoined pulses.

In some embodiments the one or more pulses are a pair of conjoined pulses configured to form a ring, a portion or ring, or a plurality of portions of a ring.

In some embodiments the one or more pulses are a plurality of pairs of conjoined pulses configured to form a plurality of rings.

In some embodiments the at least one pulse is configured to modify (e.g., increase or decrease) the RIQ for myopia control.

In some embodiments the at least one pulse is configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).

In some embodiments the at least one pulse includes a pulse located in the center of the lens.

In some embodiments the at least one pulse is created on a portion of the lens in a non-radial direction (e.g., to create an asymmetrical, non-annular, and/or non-concentric pulse).

In some embodiments the at least one pulse varies across a particular dimension of the pulse (e.g., any combination of one or more of a width, height, and/or shape varies in a particular dimension and/or direction). In some embodiments, the shape of the pulse may include parallel or non-parallel side and a planar or non-planar top.

In some embodiments, the ophthalmic lens incorporating the base power profile, the ophthalmic lens incorporating both the base power profile and the pulse elements, only the portions incorporating the pulse elements, or any part of the ophthalmic lens incorporating the base power profile and the pulse elements may be manufactured in parts or fully using at least one of lathing, molding, spinning, printing, etching, sputtering processes.

In some embodiments the ophthalmic lens provides a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, +3.1D, ±3.2D, and/or 3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.4, 0.41, 0.42, 0.43, 0.44, 0.45, 0.46, 0.47, 0.48, 0.49, or 0.5).

In some embodiments the ophthalmic lens provides a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, +3.1D ±3.2D, and/or ±3.25D)), and wherein an RIQ area of the one or more independent peaks is about 0.16 Units*Diopters (e.g., 0.1, 0.11, 0.12, 0.13, 0.14, 0.15, 0.16, 0.17, 0.18, 0.19, 0.2, 0.21, 0.22, 0.23, 0.24, or 0.25) or less.

In some embodiments the ophthalmic lens provides a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, +3.1D, ±3.2D, and/or ±3.25D)).

In some embodiments the at least one pulse is about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).

In some embodiments the at least one pulse comprises a plurality of narrow and/or annular concentric optical zones located on at least one of the front surface and/or the back surface and/or within the matrix of the ophthalmic lens and formed by line curvatures.

In some embodiments the ophthalmic lens is one of a contact lens, an intraocular lens, and/or a spectacle lens.

Other features and advantages of the subject matter described herein will be apparent from the description and drawings, and from the claims

### BRIEF DESCRIPTION OF THE DRAWINGS

Aspects of the embodiments described herein may be understood from the following detailed description when read with the accompanying figures.

FIG. 1 illustrates an exemplary embodiment of an ophthalmic lens incorporating an exemplary optical design in accordance with some embodiments described herein.

FIG. 2 illustrates an exemplary embodiment of an ophthalmic lens incorporating an exemplary optical design in accordance with some embodiments described herein.

FIG. 3 illustrates an exemplary embodiment of an ophthalmic lens incorporating an exemplary optical design in accordance with some embodiments described herein.

FIG. 4 illustrates an exemplary embodiment of an ophthalmic lens incorporating an exemplary optical design in accordance with some embodiments described herein.

FIG. 5 illustrates an exemplary embodiment of an ophthalmic lens incorporating an exemplary optical design in accordance with some embodiments described herein.

FIG. 6 illustrates an exemplary embodiment of an ophthalmic lens incorporating an exemplary optical design in accordance with some embodiments described herein.

FIG. 7 illustrates an exemplary embodiment of an ophthalmic lens incorporating an exemplary optical design in accordance with some embodiments described herein.

FIG. 8 illustrates an exemplary embodiment of an ophthalmic lens incorporating an exemplary optical design in accordance with some embodiments described herein.

FIG. 9 illustrates an exemplary embodiment of an ophthalmic lens incorporating an exemplary optical design in accordance with some embodiments described herein.

FIG. 10 illustrates an exemplary embodiment of an RIQ for the ophthalmic lens of FIG. 9.

FIG. 11 illustrates an exemplary embodiment of an ophthalmic lens incorporating an exemplary optical design in accordance with some embodiments described herein.

FIG. 12 illustrates an exemplary embodiment of an RIQ for the ophthalmic lens of FIG. 11.

FIG. 13 illustrates an exemplary embodiment of an ophthalmic lens incorporating an exemplary optical design in accordance with some embodiments described herein.

FIG. 14 illustrates an exemplary embodiment of an RIQ for the ophthalmic lens of FIG. 13.

FIG. 15 illustrates an exemplary embodiment of an ophthalmic lens incorporating an exemplary optical design in accordance with some embodiments described herein.

FIG. 16 illustrates an exemplary embodiment of an ophthalmic lens incorporating an exemplary optical design in accordance with some embodiments described herein.

FIG. 17 illustrates an exemplary embodiment of an RIQ for the ophthalmic lens of FIG. 16.

FIG. 18 illustrates an exemplary embodiment of an ophthalmic lens incorporating an exemplary optical design in accordance with some embodiments described herein.

FIG. 19 illustrates an exemplary embodiment of an RIQ for the ophthalmic lens of FIG. 18.

FIG. 20 illustrates an ophthalmic lens.

FIG. 21 illustrates an exemplary embodiment of an RIQ for the ophthalmic lens of FIG. 20.

FIG. 22 illustrates an exemplary embodiment of an ophthalmic lens incorporating an exemplary optical design in accordance with some embodiments described herein.

FIG. 23 illustrates an exemplary embodiment of an RIQ for the ophthalmic lens of FIG. 22.

FIG. 24 illustrates an exemplary embodiment of an ophthalmic lens incorporating an exemplary optical design in accordance with some embodiments described herein.

FIG. 25 illustrates an exemplary embodiment of an ophthalmic lens incorporating an exemplary optical design in accordance with some embodiments described herein.

FIG. 26 illustrates an exemplary embodiment of an ophthalmic lens incorporating an exemplary optical design in accordance with some embodiments described herein.

FIG. 27 illustrates an exemplary embodiment of an ophthalmic lens incorporating an exemplary optical design in accordance with some embodiments described herein.

FIG. 28 illustrates an exemplary embodiment of an ophthalmic lens incorporating an exemplary optical design in accordance with some embodiments described herein.

FIG. 29 illustrates an exemplary embodiment of an ophthalmic lens incorporating an exemplary optical design in accordance with some embodiments described herein.

FIG. 30 illustrates an exemplary embodiment of an ophthalmic lens incorporating an exemplary optical design in accordance with some embodiments described herein.

FIG. 31 illustrates an exemplary embodiment of an ophthalmic lens incorporating an exemplary optical design in accordance with some embodiments described herein.

FIG. 32 illustrates exemplary properties of pulses that may be modified to obtain a desirable profile and/or RIQ reduction in accordance with some embodiments described herein.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The subject headings used in the detailed description are included for the ease of reference of the reader and should not be used to limit the subject matter found throughout the disclosure or the claims. The subject headings should not be used in construing the scope of the claims or the claim limitations.

The terms “about” as used in this disclosure is to be understood to be interchangeable with the term approximate or approximately.

The term “comprise” and its derivatives (e.g., comprises, comprising) as used in this disclosure is to be taken to be inclusive of features to which it refers, and is not meant to exclude the presence of additional features unless otherwise stated or implied.

The term “myopia” or “myopic” as used in this disclosure is intended to refer to an eye that is already myopic, is pre myopic, or has a refractive condition that is progressing towards myopia.

The term “presbyopia” or “presbyopic” as used in this disclosure is intended to refer to an eye that is has a diminished ability to focus on intermediate and near objects.

The term “ophthalmic lens” or “ophthalmic device” as used in this disclosure is intended to include one or more of a contact lens, or an intraocular lens, a spectacle lens, a spectacle lens precursor (e.g., a semi-finished lens or a fully finished lens), an orthokeratology lens, an intracorneal lens or a refractive surgery light energy algorithm designed to reshape a cornea, or a film (or one or more layers).

The term “base power profile” may be a power profile of constant power or may incorporate at least one or more powers (e.g., a base power may have a power profile used to correct a single vision refractive error or to provide a defocus of the refractive error). A base power profile may be a progressive power profile where the power is constantly changing (e.g., increasing or decreasing or a combination of increasing or decreasing) in a monotonic or periodic or non-monotonic or aperiodic manner across the optical zone. The power profile may be a bifocal or trifocal or multifocal, a dual focus, progressive, toric, toric multifocal, or higher order aberrations or any combinations thereof. The base power profile may be formed by curvatures that may be spherical or line or flat or not spherical or curvatures that may be defined by any mathematical function or may be coaxial or non-coaxial (e.g., spheroidal torus' or non-spheroidal torus' and the like) or as otherwise structured to form on axis and/or off axis focal points. A base power profile may be a pre-pulsed power profile (e.g., a base power that does not contain a pulse) or it may be a power profile where only parts or all of the power profile already include pulse elements.

In some embodiments, the method, device, system or feature to correct and/or treat refractive errors and conditions of the eye may comprise ophthalmic lens designs that include one or more pulses that control an optical wavefront based on the configuration of the pulse's power (height), shape, width, location, number, tilt, and/or optical effect. In some embodiments, the interaction between the zones may also affect the formation of light inside the human eye to alter vision, alter the path of light rays, and/or control myopia as may be desired. In some embodiments, the interaction between the zones may also correct vision and/or treat vision and/or improve ocular conditions or visual conditions and disorders. In some embodiments, the interaction between the zones may also control the interference caused by one or more first focal points on one or more second focal points and/or the distribution of focal points on and off the optical axis.

In some embodiments, the method, device, system or feature to correct and/or treat refractive errors and conditions of the eye may comprise ophthalmic lens designs that include a base power profile; and at least one pulse selected to modify the base power profile, wherein the at least one pulse is selected to create a power profile with much greater precision than possible with other lens design techniques by using the one or more pulses to create an optical design configured to provide a higher resolution specification of a power profile.

In some embodiments, the method, device, system or feature to correct and/or treat refractive errors and conditions of the eye may comprise ophthalmic lens designs that include a base power profile; and at least one pulse selected to modify the base power profile, wherein the one or more pulses may be introduced to a base power profile to modify (e.g., increase, decrease, shift, redistribute, eliminate or create) one or more RIQ values (e.g., peak RIQ values) at any desired vergence along an RIQ curve. For example, in some embodiments, a bifocal base power profile may have two RIQ peaks—one at a distance vergence and one at a near vergence and one or more pulses may be introduced that create a new RIQ peak at an intermediate vergence to create a trifocal ophthalmic lens. In some embodiments, this design may also target the redistribution of a defined light energy away from the two bifocal RIQ peaks and/or a plurality of pulses may be designed to introduce multiple spaced apart RIQ peaks or a broader continuous RIQ region between the distance and near RIQ peaks of the base power to create an extended depth of focus (EDOF) while modifying (e.g. reducing) the light energy directed to the two bifocal peaks formed by the conventional pulses.

In some embodiments, the method, device, system or feature to correct and/or treat refractive errors and conditions of the eye may comprise ophthalmic lens designs that include a base power profile; and at least one pulse selected to modify the base power profile, wherein the one or more pulses may be introduced to a base power profile (e.g. a multifocal base power profile formed without pulses) based on the simultaneous vision principle and the one or more pulses may be configured to selectively modify an RIQ at any vergence (e.g. increase, decrease, eliminate, shift, redistribute, and/or create peaks, across the useful range of vergences for vision). In some embodiments, the one or more pulses may reduce the light energy of the RIQ plot at the selected vergences to a relatively low level while maintaining it sufficiently high for good vision at other vergences (e.g., distance, intermediate, near and/or anywhere in between). In some embodiments, the one or more pulses may be configured so that the undesired light energy is spread across a range of vergences (e.g., outside the useful range of vergences for presbyopic vision) In some embodiments, the one or more pulses may be configured to modify interference from a first light energy on a first image plane (e.g., increase, reduce, substantially reduce and/or minimize the interference). In some embodiments, the light energy is widely spread and of relatively low energy at the vergences (e.g. a low RIQ) including within the useful range of vergences.

In some embodiments, the pulses may be aberration free (or substantially aberration free) and/or have zero (or substantially zero) spherical aberration locally inside the pulse signal. However, different forms of pulses may be added in some embodiments (e.g., pulses with higher order aberrations, coaxial and/or non-coaxial optics).

In some embodiments, a pulse may be a single burst (e.g., a rapid change) of power with a predefined power (height), shape, width, location, and/or number that impacts retinal image quality (RIQ) (e.g., relative to base power of the ophthalmic lens). In some embodiments, the impact may be a increase and/or decrease in RIQ. In some embodiments, a single pulse may not have a significant impact on RIQ by itself but, collectively, a plurality of pulses may enable RIQ modification (e.g., redistribute, increase and/or decrease). In some embodiments, the one or more pulses may reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%. In some embodiments, a single pulse may have an impact on RIQ. For example, the single pulse may enable RIQ reduction. In some embodiments, the pulse may modify (e.g., increase and/or decrease) the RIQ (e.g., the peak RIQ and/or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%. In some embodiments, the peak RIQ may decrease but the RIQ in other locations may increase along the y-axis. In some embodiments, the increase of RIQ at other (non-peak) vergences may be configured to provide depth of focus and/or improved vision for presbyopia patients. In some embodiments, the one or more pulses may be configured to modify the RIQ height, shape, and/or area under the curve generally and/or at specific targeted vergences anywhere along the through focus plot (e.g., +/−1 D, +/−2 D, +/−3 D, +/−5 D, +/−10 D, or +/−20 D). In some embodiments, the modification of RIQ may benefit vision correction or vision treatment or ocular vision conditions such as e.g., increasing, reducing and/or minimizing interference by first images on second images, increasing (e.g., introducing peaks) or decreasing RIQ at targeted vergences, and/or altering the RIQ shape. In some embodiments, the one or more pulses may be configured to alter the RIQ shape and/or spread light energy widely and/or redistribute the RIQ shape along one or more vergences to provide e.g., an EDOF, a treatment of refractive errors, improvement of night vision, and/or enhance or reduce certain optical effects (e.g., instability of vision with blinking, coma, flare, haloes, positive and/or negative dysphotopsias).

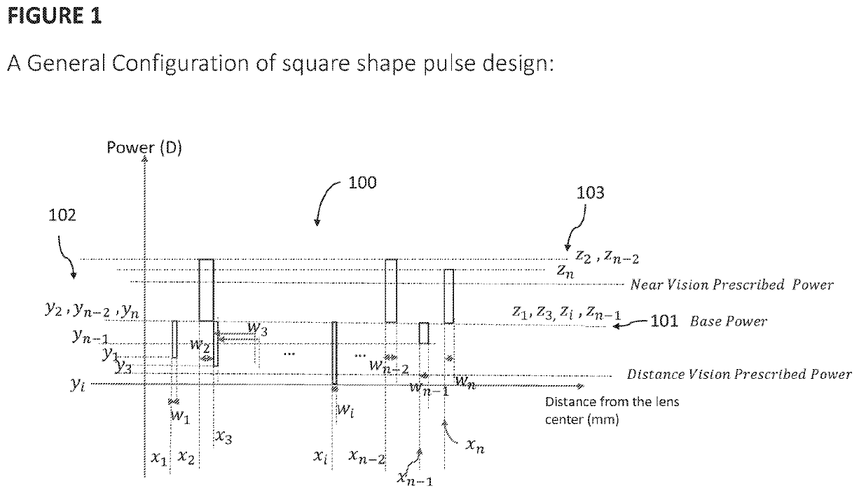

FIG. 1 illustrates an exemplary embodiment of an ophthalmic lens incorporating an exemplary optical design in accordance with some embodiments described herein. As illustrated, the ophthalmic lens may have a base power profile (e.g., a single vision base power but not powered to correct a distance and/or near refractive error) and a plurality of pulses. The pulses may be characterized as having a rapid change of relatively more positive or more negative power, a predetermined width, and a predetermined height. In some embodiments, the ophthalmic lens may comprise one or more pulses (e.g., 1, 2, 3, 4, 5, 6, 7, 8, 9, 10, 11, 12, 13, 14, 15, 20, 25, 30, 35, 40, 45, 50, 60, 70, 80, 90, or 100 pulses). In some embodiments, the pulses may have the same or different widths and or the same or different heights (e.g., power). For example, one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, 0.25-0.3 mm). For example, the one or more pulses may have a height (e.g., dioptric power) of about +/−0.25 to 30 D (e.g., +/−0.25 D, +/−0.50 D, +/−0.75 D, +/−1 D, +/−1.25 D, +/−0.50 D, +/−1.75 D, +/−2 D, +/−2.25 D, +/−2.50 D, +/−2.75 D, +/−3 D, +/−3.25 D, +/−3.50 D, +/−3.75 D, +/−4 D, +/−5 D, +/−6 D, +/−7 D, +/−8 D, +/−9 D, +/−10 D, +/−12.5 D, +/−15 D, +/−17.5 D, +/−20 D, +/−25 D, +/−30 D, +/−0.25-1 D+/−1-5 D, +/−5-10 D, +/−10-20 D, +/−20-30 D. In some embodiments the at least one pulse has a height (e.g., a dioptric power) of between −100 D to +100 D (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D). In some embodiments, the one or more pulses may be formed on one or more of the front surface of the ophthalmic lens and/or the back surface of the ophthalmic lens and/or within the matrix of the ophthalmic lens.

As shown in the embodiment of FIG. 1, the ophthalmic lens may include n pulses in the ophthalmic lens power profile 100, where n can be any number from e.g., 0 to 100. The pulses may have a lower power value (e.g., relative to the base power of the lens) that is identified as the y value 102, and/or a higher power value (e.g., relative to the base power of the lens) that is identified as Z value 103. A pulse in this example starts at an x value and has a width of w. For example, the first pulse may start at x1 along the x axis showing the distance from the ophthalmic lens center. In general, FIG. 1 describes the optical power profile of an ophthalmic lens (e.g., a base power profile 101 modified by pulses) where the pulses are defined using different mathematical variables. Configurations of pulse designs with varying parameter values, such as those illustrated in FIG. 1, can be used for optimization of ophthalmic lens designs. Accordingly, this embodiment describes certain design variables that a designer may use to create a desirable lens. As illustrated, the parameter n represents the number of pulses across the optical zone. In some embodiments, n may be relatively large such that the lens design includes (or is capable of including) a high frequency of very fine pulses to provide a high resolution specification of powered pulses to control the optical design of an ophthalmic lens. In some embodiments, n can be any value up to 30, 50, 70 or 100 or more.

Additionally, in some embodiments, the near/distance vision power prescribed for a patient may be different to the different levels of power for the pulses compared to the base power. In some embodiments, the power of a pulse may not be limited to the power of a conventional part of and/or all of a power profile with no pulses that may be prescribed by a practitioner to correct the refractive error. For example, a near reading addition for advanced presbyopes may require an add power of +2.5 or +3.0 D however the pulses may be configured more desirably as +5.0D for 3 narrow pulses and +1.0D for 2 pulses and −4.0D for 3 pulses. In some embodiments, the greater number of more finely specified and higher resolution pulses may modify the RIQ more precisely and provide an improved vision correction (e.g., a longer depth of focus and/or modified interference of out of focus light on the in-focus images).

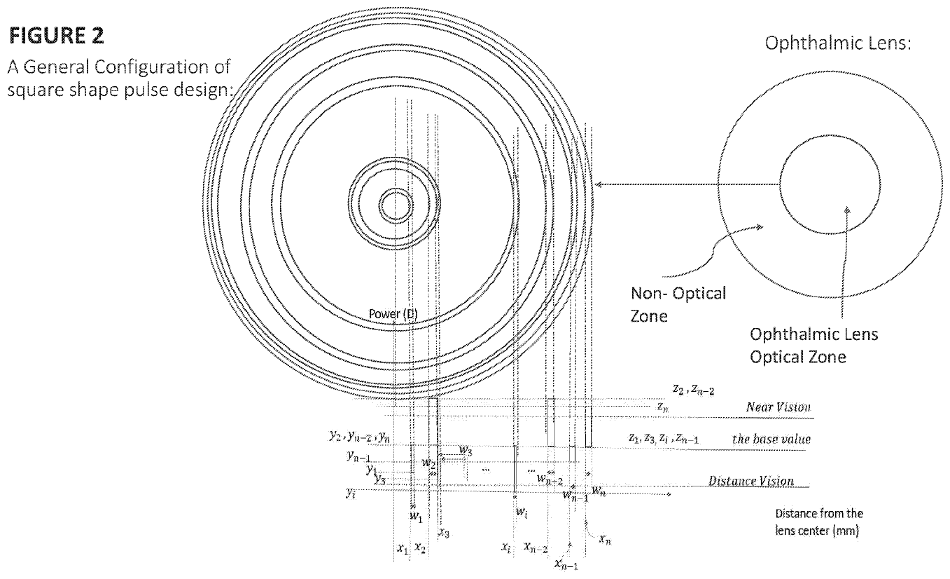

FIG. 2 illustrates plan views of an exemplary embodiment of an ophthalmic lens incorporating an exemplary optical design in accordance with some embodiments described herein. This figure illustrates the distribution of the pulses within the optical zone formed on the front surface of the example ophthalmic lens described in in FIG. 1. As illustrated in this embodiment, the center optical zone includes a base power more positive in power than a distance vision refractive error correction and less positive than a near vision refractive error correction and four concentric annular relatively more negative powered pulses and three relatively more positive powered pulses than the base power. In some embodiments, the ophthalmic lens may include an equal number of pulses e.g., for combinations of one or more of relatively more positive and/or more negative powers. In some embodiments, the ophthalmic lens may include different numbers of pulses for distance and near vision (e.g., more distance vision pulses or more near vision pulses). In some embodiments, the individual pulses may not be configured for near and/or distance vision but the combination of pulses (e.g., relatively more positive and/or negative powered pulses) may provide acceptable vision for distance, near and/or intermediate distances (e.g., provide a depth of focus). In some embodiments, the ophthalmic lens may have one or more relatively more positive and/or negative pulses.

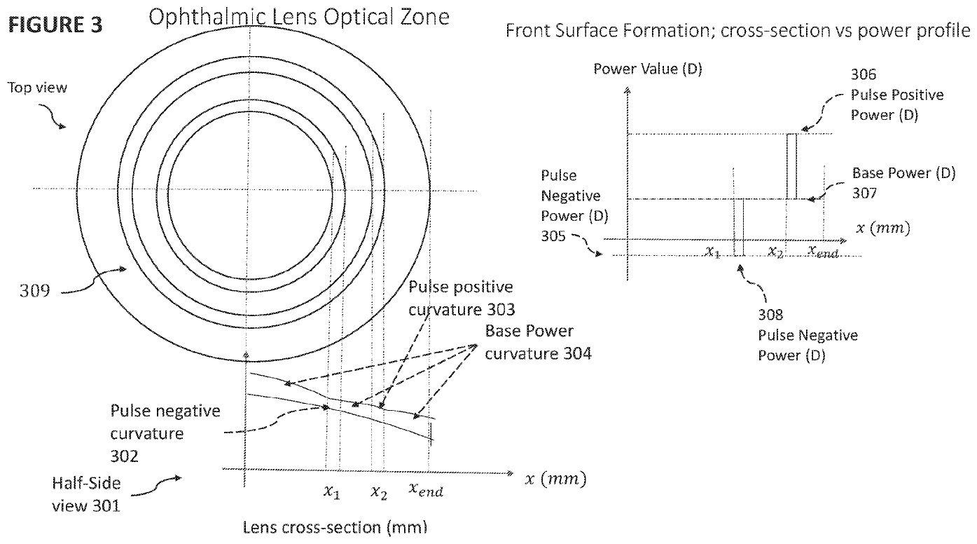

FIG. 3 illustrates plan and cross sectional views of the central optical zone of an exemplary embodiment of an ophthalmic lens incorporating an exemplary optical design in accordance with some embodiments described herein. The plan view shows two concentric annular pulses located within the optical zone and the corresponding power profile of the base power and the two pulses e.g., one relatively more negative pulse 308 and one relatively more positive pulse 306 than the base power 307. FIG. 3 illustrates various embodiments of surface curvature to achieve the pulses described herein. FIG. 3 also illustrates a cross section 301 of the ophthalmic lens including the front surface curvatures forming the pulses. In this example, the surface change is shown in a side view 301. As illustrated, the negative pulse curvature 302 is flatter than the base power curvature 304, and the positive pulse curvature 303, is steeper than the base power curvature 304. As further illustrated in FIG. 3, the surface of 309 is a full optical surface that is continuous (e.g., is free of transitions and/or blends). In some embodiments, there may not be a non-optical blending zone. In some embodiments, a non-optical blending zone may be present (e.g., if the manufacturing process requires one because e.g., it cannot avoid producing a blend or transition of the non-optical blending portion because the tool tip radius is too large for the rapid change in power characteristics of a pulse). In some embodiments, the non-optical blending zone may be continuous and/or discontinuous. In some embodiments, the lens surface may be continuous and/or may not require blending zones when both positive and negative pulses are present.

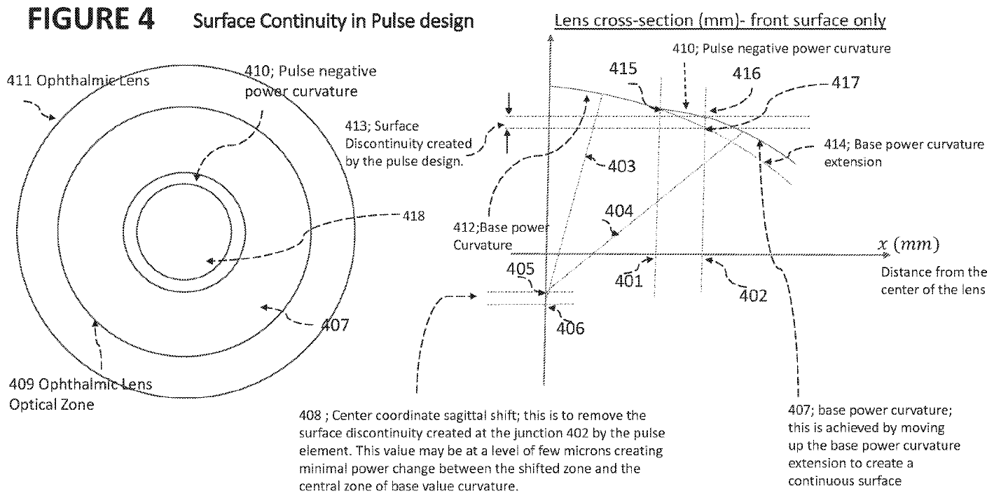

FIG. 4 illustrates an exemplary embodiment of an ophthalmic lens incorporating an exemplary optical design in accordance with some embodiments described herein. FIG. 4 shows an example of an ophthalmic lens 411, that has an optical zone 409 with three zones 418, 410 and 407 and illustrates a technique to create a continuous surface while adding a pulse on the surface of the lens. The embodiment in FIG. 4 illustrates one approach to forming a continuous surface (e.g., without a transition or blending or non-optical blending zone, between the curvatures required to form the rapid power change of a pulse and the base surface). As shown, the curvature forming the negative powered pulse 410 connects to the base power curvature 412 at a location 401 along the horizontal axis forming a continuous surface at the junction point 415. The other end of the pulse at 402 along the x-axis, may not be continuous with the base power curvature 414 at 417. As a result, to make the surface continuous, the base power curvature 414 may be moved to meet the curvature 410 at 416 and create a continuous surface. Shifting the intersection of the curvatures from 417 to 416, may require the center of the curvature 414 to move accordingly from 406 to 405 for that section of the lens, as drawn at 408. In some embodiments, the shift of the base power curvature 414 may be very small (e.g., at a micron level) and may not create any significant optical power difference with the central portion 418 and the base power portion 407 and therefore may not affect the wearer's vision at a specific vergence (e.g., may not substantially impact the RIQ at any vergence and therefore, not affect the wearer's vision).

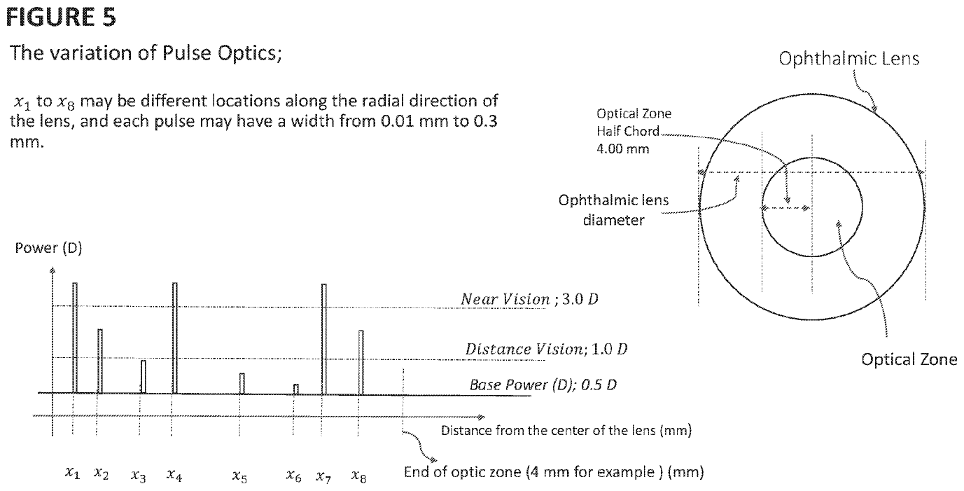

FIG. 5 illustrates an exemplary embodiment of an ophthalmic lens (e.g., a presbyopic lens) incorporating an exemplary optical design in accordance with some embodiments described herein. As illustrated in this embodiment, the plurality of pulses may have varying powers relative to the base power profile. In some embodiments, all of the pulses may be relatively more positive in power than the base power or may be relatively more negative than a base power or may include any combination of relatively more positive and negative pulses than the base power profile. In the example illustrated in FIG. 5, the pulses x₁ to x₈ are all positive relative to the base power and the base power (+0.5D) is less positive than both the power required to correct the distance refractive error (+1.0D) and near refractive error (+3.0D). This example illustrates that the distance vision and near vision powers may be different to the pulse power levels. In some embodiments, the base power profile may correspond to the power used to correct a distance refractive error or an intermediate refractive error or a near refractive error or any combination thereof and the pulses may have a plurality of heights (e.g., powers) that may or may not correspond to the base power required to correct the refractive error of the wearer at any particular vergence. Accordingly, more generally, the pulse power levels may simply be described as differing (e.g., relatively more positive or negative) relative to the base power profile.

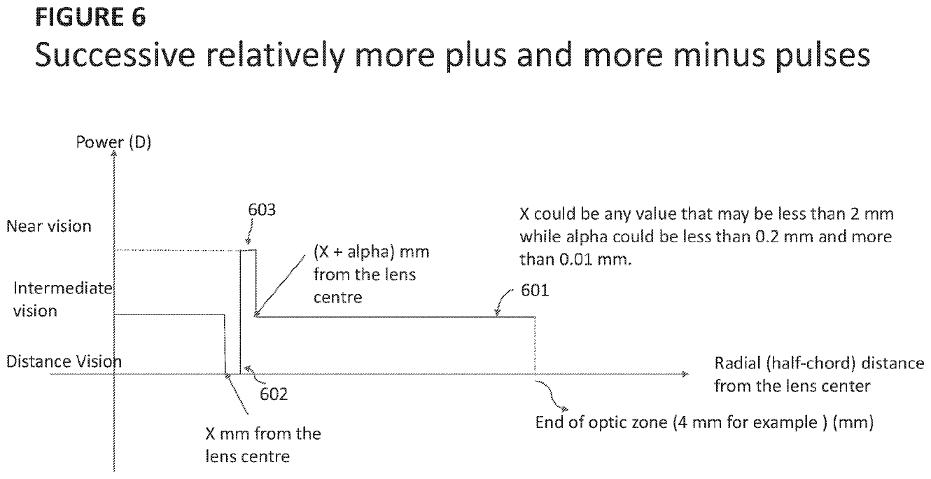

FIG. 6 illustrates an exemplary embodiment of an ophthalmic lens (e.g., a presbyopic lens) incorporating an exemplary optical design in accordance with some embodiments described herein. The embodiment illustrated in FIG. 6 is an example of an ophthalmic lens with a base power profile 601 and a relatively more negative pulse 602 followed by a relatively more positive pulse 603 than the base power profile. In some embodiments, the base power profile may be selected to be useful for intermediate vision and the more negative pulse may be useful for distance vision while the more positive pulse may be useful for near vision. As illustrated, in some embodiments, the pulses 602 and 603 may be conjoined (e.g., form two adjacent concentric annular rings). In some embodiments, two or more pulses may be conjoined and the pulses may include any combination of relatively more positive pulses and/or more negative pulses in any sequence or order with the same or different pulse parameters including but not limited to height (D), width, shape, tilt, and/or location. In some embodiments, a design such as the one illustrated with defined variables may provide good near, intermediate and distance vision using only two pulses—e.g., a relatively more positive and a more negative pulse conjoined with one another as illustrated. In some embodiments, a conjoined design with a vision signal with a lower intensity of rays may be enough for the eye to perceive acceptable vision for distance and near objects. For example, the two conjoined pulses of equal and opposite power described in this embodiment and formed on an intermediate base power profile may provide a trifocal optical effect with the intermediate focal point located between the 2 pulse focal points and the more negatively powered pulse relative to the base intermediate power may provide a focal point on the retina (e.g., the fovea) for distance vision while the relatively more positive pulse may form a focal point more anterior to the intermediate focal point (e.g., an equidistance from the intermediate focal point as the foveal focal point) for near vision in the presbyopic eye. In some embodiments, the light energy of the focal points formed by the two pulses may be of relatively low energy compared to a non-pulse refractive element and as such may form only a very small RIQ peak at the distance vision vergence and the near vision vergence. The small RIQ peaks may be sufficiently large to provide good distance vision and good near vision e.g., when out of focus light from focal points formed by the low energy light rays provide modified interference (e.g., increased, decreased, and/or minimized) relative to in-focus images at near and distance vergences. In some embodiments, the intermediate focal point may be located at approximately the midpoint of the distance between the pulse focal points (or alternatively at any point between the focal points formed by the pulses). In some embodiments, a plurality of pulses may be configured in the optical zone and on an intermediate base power profile and form a substantially continuous number of low energy focal points on either side of the intermediate focal point of the base power and form an extended depth of focus.

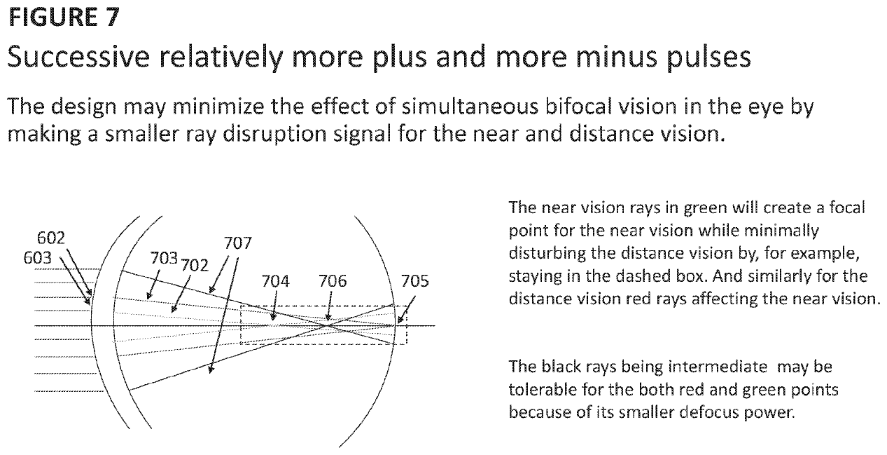

FIG. 7 illustrates an exemplary embodiment of an ophthalmic lens incorporating an exemplary optical design in accordance with some embodiments described herein. FIG. 7 corresponds to the embodiment illustrated in FIG. 6 and illustrates how the parameters of the pulses 602 and 603 may be designed to direct a small bundle of light energy 702, 703 to form low energy focal points 704 and 705 that may reduce/minimize the interference effect of those low energy light rays from the pulses forming a trifocal optical effect in conjunction with the intermediate light rays 707 and focal point 706 formed by the base power thereby providing good vision. In some embodiments, a very low intensity signal may be enough for human eye to vision at a specific vergence (such as near vision or distance vision) and such a signal may have no/small out of focus destructive effect that may disturb vision at other vergences. (e.g., there may be no RIQ change but a change in the vision may occur).

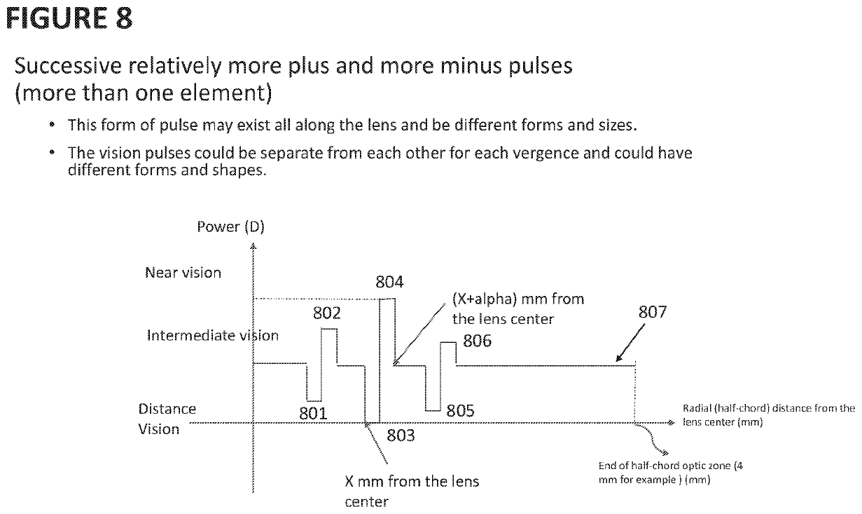

FIG. 8 illustrates an exemplary embodiment of an ophthalmic lens (e.g., a presbyopic lens) incorporating an exemplary optical design in accordance with some embodiments described herein. This embodiment illustrates multiple pairs of pulses 801-802, 803-804, 805-806 to form multiple rings (e.g., a plurality of conjoined annular concentric rings such as the conjoined pulses illustrated in FIG. 6). In some embodiments, the pulses may include successive pulses e.g., 801-802 and 805-806, successive conjoined pulses e.g., 803-804, a combination of relatively more positive and more negative pulses than the base power profile 807 spaced apart (e.g., randomly spaced, regularly spaced or spaced according to a predefined pattern or mathematical function). In some embodiments, a pulse parameter may be similarly specified including but not limited to height, width, spacing, tilt, shapes, relative power direction. In some embodiments, the design may or may not include off-axis focal points (formed by the base power profile and/or formed by the pulses). In some embodiments, the pulse designs described herein may be combined with other lens technologies. In some embodiments, other lens technologies may include other optical principles or other lens design technologies (e.g., the use of refractive elements, non-refractive elements, diffracting elements, light scattering elements, higher order aberrations, EDOF etc.). Additionally, as illustrated in more detail below, the techniques described herein may be used to alter the RIQ and different optical wavefronts for vision correction e.g., presbyopia correction and/or vision treatments e.g., myopia control, and or any other vision conditions.

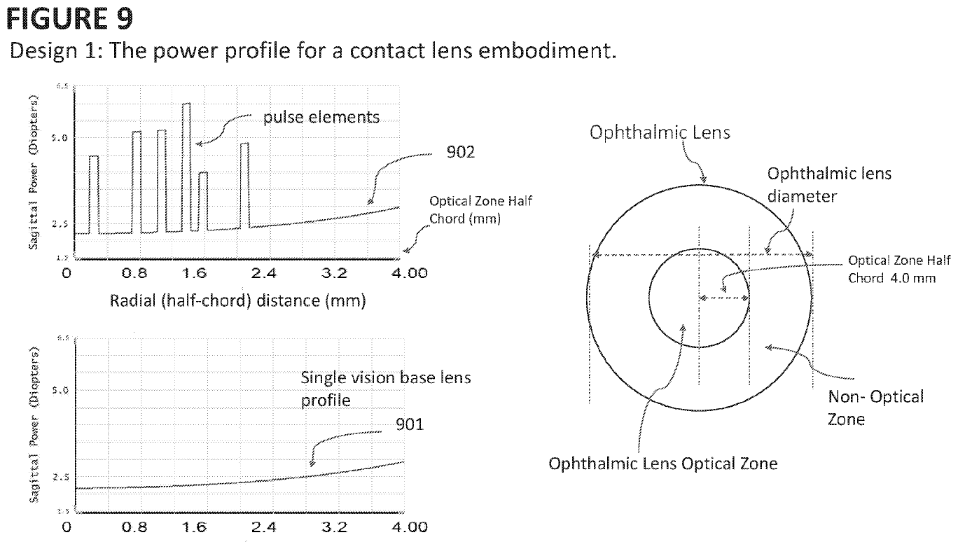

FIG. 9 illustrates an exemplary embodiment of an ophthalmic lens incorporating an exemplary optical design in accordance with some embodiments described herein. As illustrated the ophthalmic lens (e.g., a contact lens) comprises a base power profile 901 and is a single vision contact lens used to correct a refractive error e.g. a distance vision refractive error. A portion of the optical zone of the base power profile 901 is modified with a plurality of pulses (e.g., 6 relatively more positive pulses than the base power profile) within a half-chord distance of 4 mm to form a modified power profile 902.

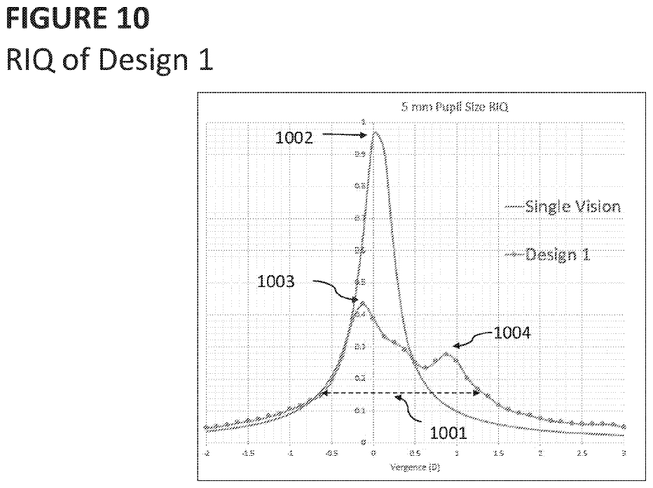

FIG. 10 illustrates an exemplary embodiment of an RIQ for the ophthalmic lens incorporating pulses of FIG. 9. As illustrated, the power profile 902 shows a modified (e.g., improved) RIQ relative to the single vision lens base power profile 901 of FIG. 9. A reduced RIQ peak and modified shape, as described earlier, may improve the lens performance to be used for vision correction (e.g., presbyopia correction and/or vision treatments (e.g., myopia control, and or any other vision conditions)). The peak RIQ of the lens design incorporating pulses in this example at about 0 vergence was reduced and is less than 0.5. In the illustrated example, the six positive pulses lowered the peak RIQ 1002 to 1003 and at the same time created a second peak RIQ 1004 at about a 1D vergence and introduces a longer depth of focus 1001 than the base power profile 901. This demonstrates the impact a plurality of pulses may have on precisely redistributing the light energy across the RIQ as compared with a conventional non-pulse lens design to provide an improved or more useful RIQ providing additional functionality for vision. In some embodiments, the one or more peak RIQ of a power profile modified by pulses may be less than 0.7, 0.6, 0.5, 0.4, or 0.3 or lower.

The exemplary embodiments of FIGS. 9 and 10 illustrate that pulses added to a single vision base lens (FIG. 9) may alter (e.g., significantly alter) the RIQ peak value 1002 in the single vision lens to a lower value 1003. In some embodiments, the lower value of the RIQ peak (by being less destructive) may provide the possibility of good vision at other vergences and/or create a depth of focus 1001. In some embodiments, the control of RIQ and image quality may have a different and finer effect than the effect of conventional non-pulse rings (or other features) which may result in a second high energy peak created in the RIQ graph (e.g., the MiSight design). In some embodiments, the lower peak of the RIQ 1003 may indicate lower contrast in the image perceived by the wearer. In some embodiments, the lower contrast may provide or assist with myopia control. In some embodiments, the reduced peak at 1004 and/or the increased depth of focus 1001 may correct vision of a presbyope and/or provide a myopic defocus for myopia control. In some embodiments, the pulses may be designed to redistribute the RIQ (e.g. lower the peak RIQ) and reduce contrast of the vision at one or more vergences. In some embodiments, the reduced peak at 1004 and/or the increased depth of focus 1001 may improve at least one condition of the eye (e.g., presbyopia, myopia, hyperopia, astigmatism, binocular vision disorders, night vision image quality and/or visual fatigue syndrome).

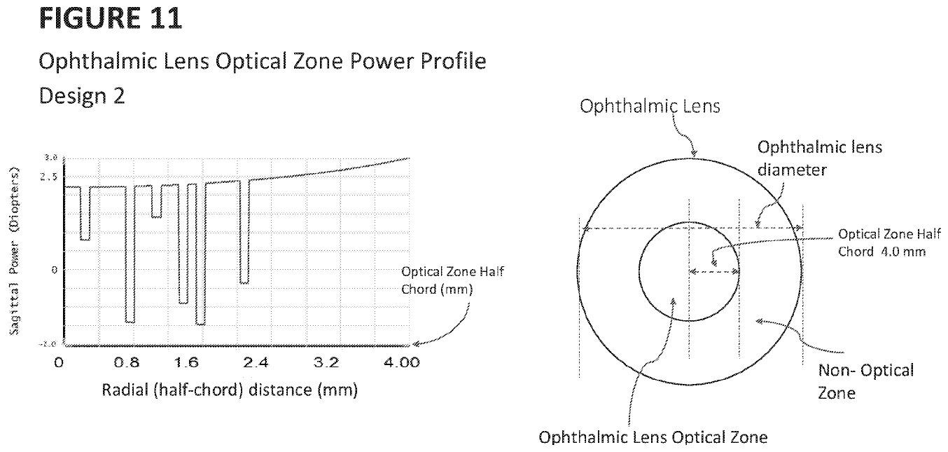

FIG. 11 illustrates an exemplary embodiment of an ophthalmic lens incorporating an exemplary optical design in accordance with some embodiments described herein. As illustrated the ophthalmic lens is a contact lens and may comprise a base power profile of a single vision lens as in FIG. 9 modified by a plurality of pulses (e.g., 6 relatively more negative pulses than the base power profile) over a half-chord distance of 4 mm.

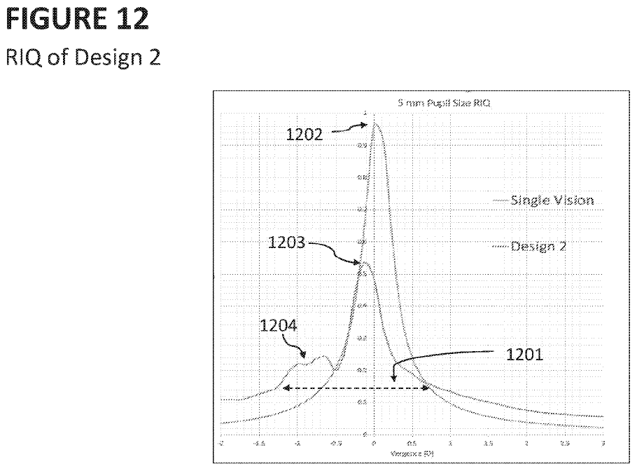

FIG. 12 illustrates an exemplary embodiment of an RIQ for the ophthalmic lens of FIG. 11. As illustrated, the lens design shows an improved or modified RIQ performance relative to a single vision lens. The peak RIQ in this example is slightly more than 0.5. In some embodiments, the peak RIQ may be about 0.7, 0.6, 0.5, 0.4, or 0.3 or lower. In some embodiments, the pulses may modify the RIQ and the modification may result in improved vision and/or myopia control. This embodiment illustrates that the pulses, using all relatively more negative pulses than the base power, as opposed to the more positive pulses in FIGS. 9 and 10, added to a single vision base lens (FIG. 11), changes (e.g., reduces or significantly reduces) the RIQ peak value 1202 in the single vision lens to a lower value 1203. The lower value of the RIQ peak (e.g., by being less destructive) may provide the possibility of vision in other vergences and/or create a depth of focus 1201. The lower peak of the RIQ data indicates lower contrast in the image perceived by the wearer that may assist with myopia control. In some embodiments, the one or more negative pulses may modify the RIQ precisely by modifying the light energy from the single vision peak to create any other desired (e.g., smaller) RIQ peaks and/or to create regions of RIQ that extend depth of focus. In some embodiments, the peaks may be created at a vergence behind the retina (e.g., negative vergence <0). For example, the new peak RIQ area 1204 at about −1 D may be refracted to the 0 vergence and hence the band of RIQ between 1204 to 1201 and the large RIQ peak 1203 may be placed in front of the retina for myopia control and/or presbyopia correction and/or alleviate visual fatigue.

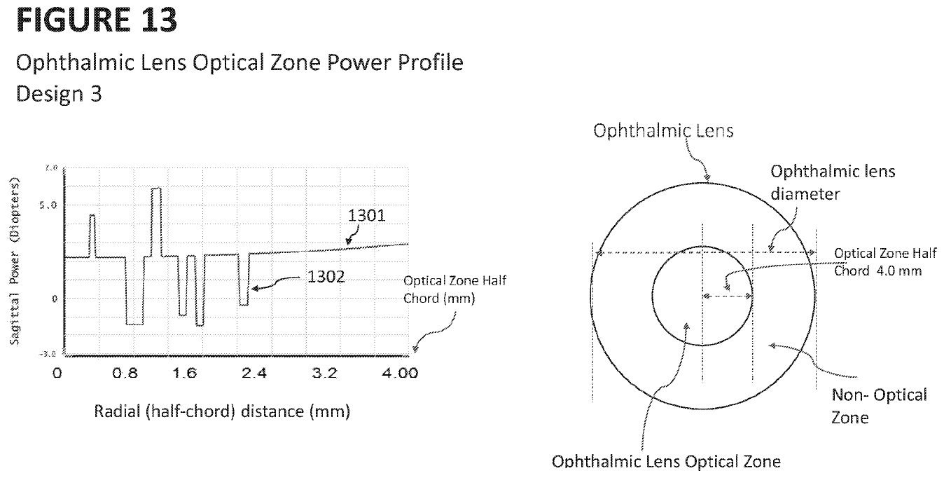

FIG. 13 illustrates an exemplary embodiment of an ophthalmic lens incorporating an exemplary optical design in accordance with some embodiments described herein. As illustrated the ophthalmic lens is a contact lens and may comprise a base power profile 1301 of a single vision lens as in FIG. 9 modified by a plurality of pulses (e.g., 2 relatively more positive and 4 relatively more negative pulses than the base power profile) within a half-chord distance of 4 mm. In this example, the pulses are spaced apart and the pulse parameters e.g., heights, widths, shapes and spacings are not constant.

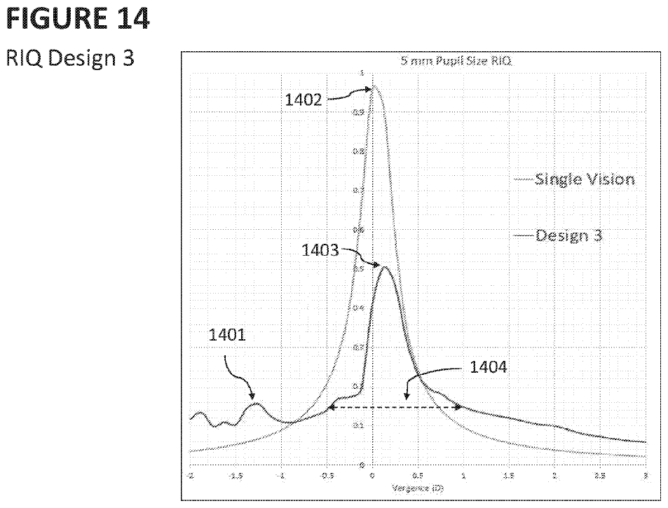

FIG. 14 illustrates an exemplary embodiment of an RIQ for the ophthalmic lens of FIG. 13. As illustrated, the lens design shows a modified (e.g., improved or changed) RIQ performance relative to a single vision lens. The peak RIQ 1403 in this example is about 0.5. This embodiment illustrates that the plurality of pulses, using a combination of relatively more negative and relatively more positive pulses of different configurations, added to a single vision lens base power profile 1301 the same as the 901 described in FIG. 9. may change (e.g., reduce or significantly reduce) the RIQ peak value 1402 of the single vision lens to a lower value 1403. The lower value of the RIQ peak (by being less destructive) may redistribute the RIQ energy across the vergences and provide minor peaks at other vergences and may enable other vision functionalities, and creates a depth of focus 1404 that may be effective for vision as a result of lowering the destructive RIQ peak in the single vision chart. In some embodiments, the lower peak of the RIQ data may indicate lower contrast in the image perceived by the wearer that may provide myopia control. In this embodiment, a secondary peak 1401 is also created and can be used for distance vision. In some embodiments, the pulses may modify the light energy and form a new RIQ profile to add functionality (e.g., as stated peak 1401 may be refracted to become 0 vergence and correct a distance refractive error and the other peaks (e.g., 1403) and RIQ band 1404 may be used for may be used for vision correction and/or treating vision disorders and/or alleviating vision conditions (e.g., myopia control). In some embodiments, a designer may introduce more pulses to further reduce the peak RIQ at 1403 to other vergences (e.g., within the vergence range useful for presbyopic vision and/or more widely outside this range to lower light energy of focal points and RIQs within the presbyopic vergence range and/or to increase, reduce or minimise the interference of out of focus light on focal planes required for vision). In some embodiments, there may be a correlation between the vision and vergences on RIQ. In some embodiments, the pulses may be configured to manipulate the out of focus light to create a depth of focus. In some embodiments, the one or more pulses may not reduce or minimize the out of focus effect. For example, in FIG. 13, none of the pulses are at the 0 to +2 power, but there may still be the depth of focus there which is a result of the defocus and focus light from all the pulses. In some embodiment, the one or more pulses may be used to selectively alter the RIQ (e.g., the number of peaks, the slope, and/or the area under the curve and the symmetry).

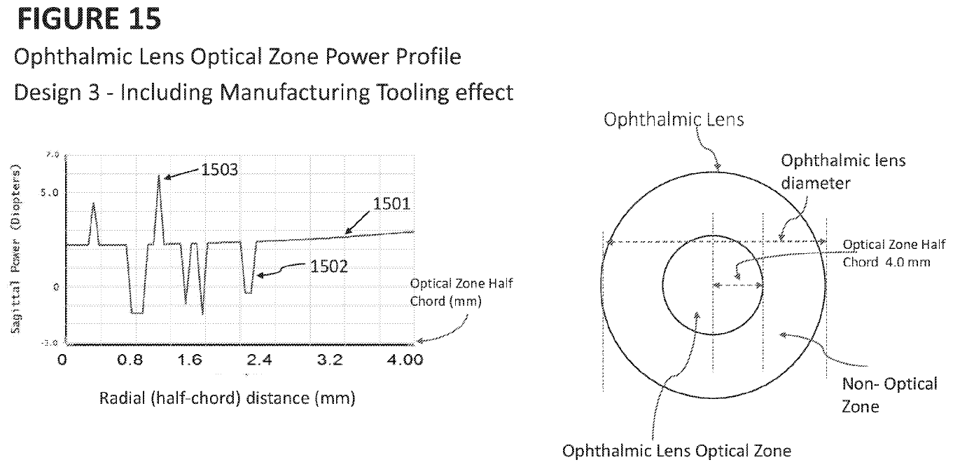

FIG. 15 illustrates an exemplary embodiment of an ophthalmic lens incorporating an exemplary optical design in accordance with some embodiments described herein. As illustrated the ophthalmic lens is a contact lens and may comprise a base power profile 1501 of a single vision lens as in FIG. 9 modified by a plurality of pulses (e.g., 2 relatively more positive and 4 relatively more negative pulses) within a half-chord distance of 4 mm. In this example, the pulses are spaced apart and the pulse parameters e.g., heights, widths, spacings and shapes are not constant. In this embodiment the sides of the pulses are not substantially parallel (e.g., not rectangular) and the peaks of the pulses are not all planar e.g. pointed and so the pulse shapes have triangular and/or trapezoidal shapes (e.g., to account for manufacturing tooling effects). In some embodiments, the shapes illustrated in this example may be the result of manufacturing methods or processes or machinery and/or limitations of metrology (at least in part) and the vertical components of the pulse 1302 (see e.g., FIG. 13) may be less vertical and not parallel sided and more like that shown in FIG. 15 at 1502 or 1503. For example, the machinery's capability to produce discrete pulses by lathing, molding, printing, stamping, laser processes and machining conditions (e.g., cutting speeds, material properties of the lens or lens precursors (tool, molds and the like), tool tip radius) may be less fine or become worn and so more rounded and more irregular peaks of the pulses may be produced. Likewise, metrology methods may be limited by resolution and averaging or smoothing algorithms (e.g., wavefront based power metrology such as NIMO). In some embodiments, manufacturing and/or metrology errors may also affect the shape and dimensions of the pulses produced or measured (e.g. sharpness of corners or tops of the power profile of the pulses) and/or the area under the power curve (see, e.g., FIG. 13) and result in a more blended transition to e.g., alter an RIQ model.

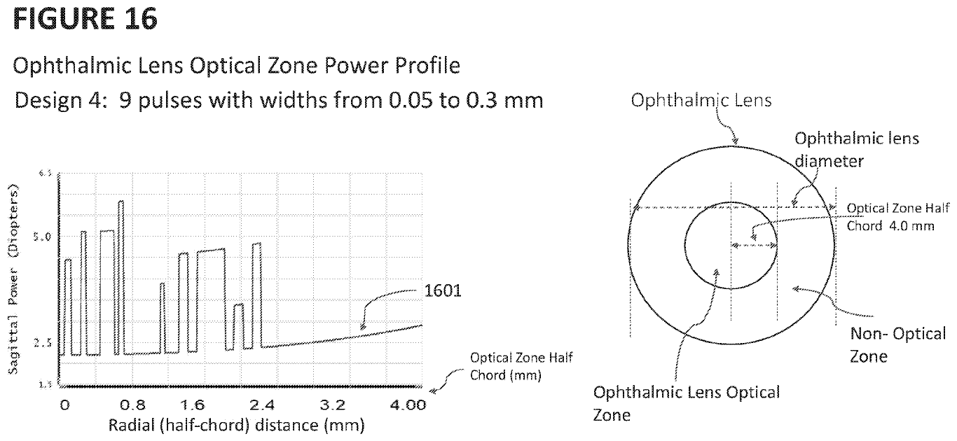

FIG. 16 illustrates an exemplary embodiment of an ophthalmic lens incorporating an exemplary optical design in accordance with some embodiments described herein. As illustrated the ophthalmic lens is a contact lens and may comprise a base power profile 1601 and a plurality of pulses (e.g., 9 relatively more positive pulses than the base power profile) within a half-chord distance of 4 mm. As further illustrated, the width of the various pulses varies from 0.05 to 0.3 mm. In some embodiments, the spacing and height (power) of the pulses may also vary.

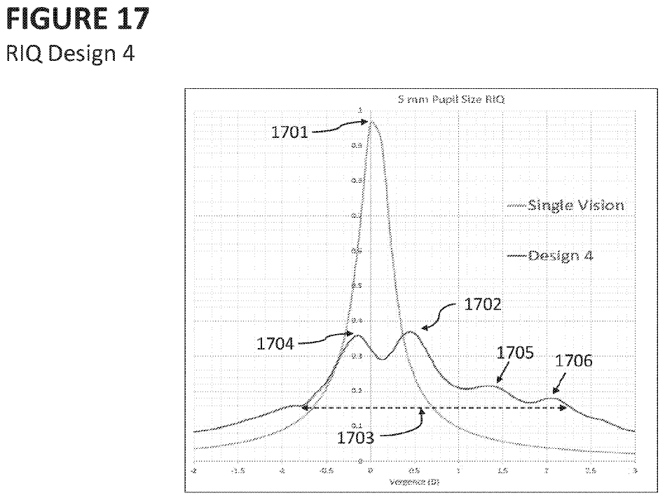

FIG. 17 illustrates an exemplary embodiment of an RIQ for the ophthalmic lens of FIG. 16. As illustrated, the lens design shows a modified (e.g., improved) RIQ performance relative to a single vision lens. The peak RIQ 1702 in this example is less than about 0.4. As illustrated in FIG. 16, the lens design used 9 pulses and the RIQ 1701 may be altered (e.g., reduced or significantly reduced) to a lower peak 1702 (e.g., by redistributing the RIQ to multiple (e.g., 4) smaller peaks; 1702, 1704, 1705 and 1706). In some embodiments, the peak RIQ may correspond to the highest value of the RIQ graph. In some embodiments, the other peaks may result in a less significant change in some vergences. In some embodiments, the lower and/or less destructive peak value compared to that of a single vision lens may also provide for increased depth of focus 1703. In some embodiments, the less destructive peak may not contribute to EDOF per se but may enable the EDOF to provide better vision. In some embodiments, the less destructive peak may modify the interference from out of focus rays at one or more vergences may be altered (e.g., minimized or substantially reduced) because of the lower RIQ profile. In some embodiments, the depth of focus may be about 3 D. In some embodiments, the number of pulses may be 2, 3, 4, 5, 6, 7, 8, 9, 10, 11, 12, 13, 14, 15, 16, 17, 18, 19, and/or 20. In some embodiments, the depth of focus may be about 0.5 D, 0.75 D, 1 D, 1.25 D, 1.5 D, 1.75 D, 2 D, 2.25 D, 2.5 D, 2.75 D, 3 D, 3.25 D, 3.5 D, 3.75 D, 4 D, 4.25 D, 4.5 D, 4.75 D, and or 5 D.

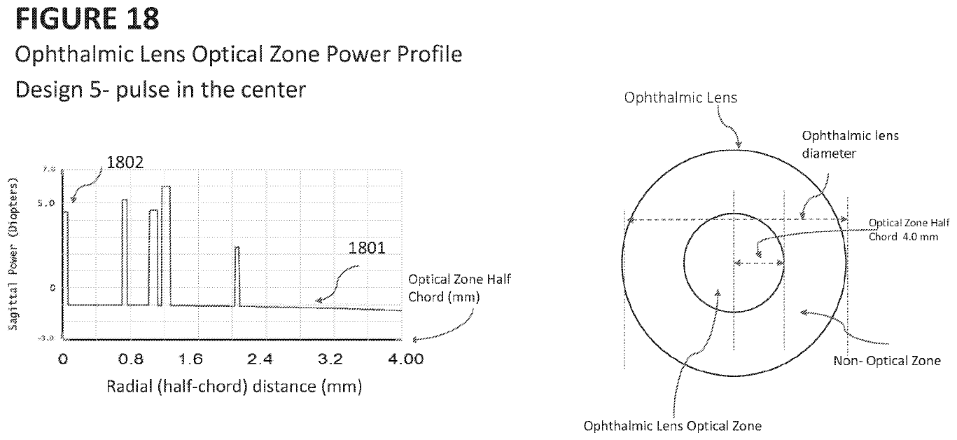

FIG. 18 illustrates an exemplary embodiment of an ophthalmic lens incorporating an exemplary optical design in accordance with some embodiments described herein. As illustrated the ophthalmic lens is a contact lens and may comprise a base power profile 1801 and plurality of pulses (e.g., 5 relatively more positive pulses than the base power) within a half-chord distance of 4 mm. As further illustrated, this design includes a pulse 1802 located in the center of the lens. As illustrated, in some embodiments, the lens profile may include a central pulse. In some embodiments, the central pulse may be a relatively more positive or a relatively more negative power than the base power and may be a conjoined pulse of the same power or a mixed power conjoined pulse element or may be a plurality of centrally located pulses with parameters or configurations similar to pulses disclosed herein (e.g., concentric, non-concentric, annular, non-annular etc.).

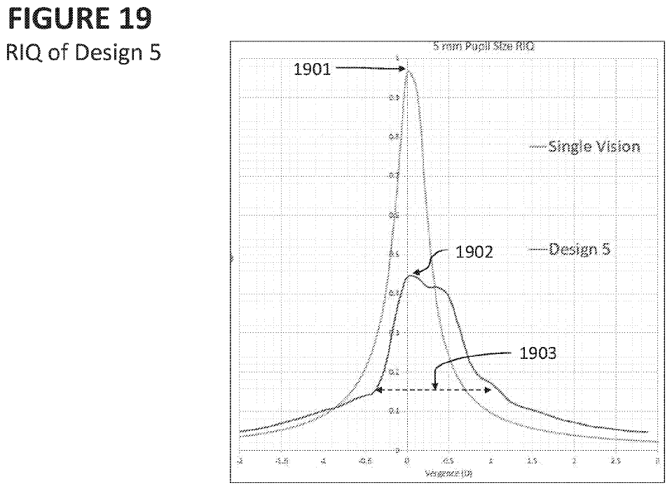

FIG. 19 illustrates an exemplary embodiment of an RIQ for the ophthalmic lens of FIG. 18. As illustrated, the lens design shows a modified (e.g., improved) RIQ performance relative to a single vision lens RIQ 1901. The peak RIQ 1902 in this example is less than about 0.5 (e.g., about 0.45). As shown in FIG. 19, the design in FIG. 18 also creates a depth of focus 1903 and the peak value of the RIQ 1902 is lower than that of a single vision lens 1901.

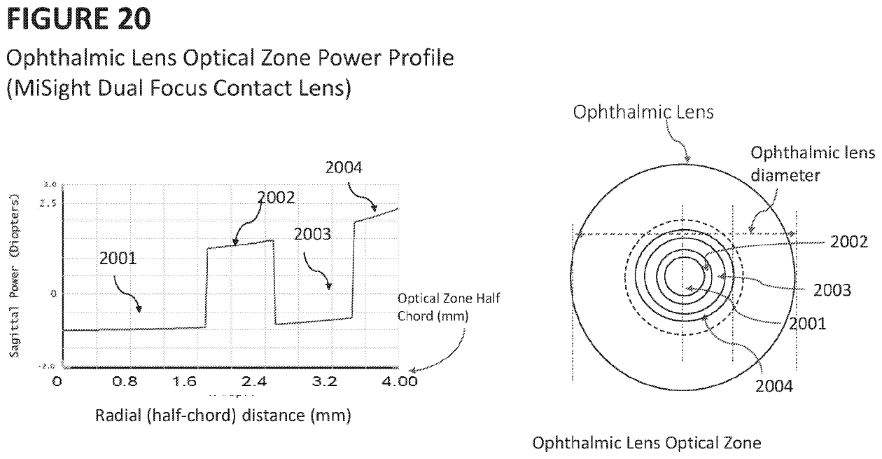

FIG. 20 illustrates an ophthalmic lens comprising a central optical zone of base power and 2 annular zones of relatively more positive power than the base power profile alternating with zones of about base power. (e.g., a commercial myopia control dual focus contact lens (Misight™, Coopervision Inc., USA)).

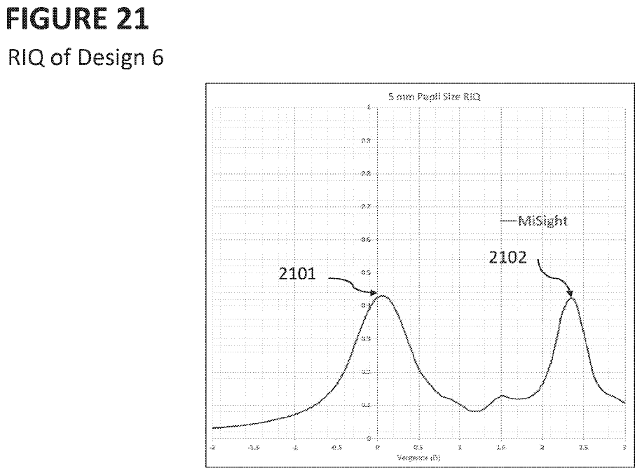

FIG. 21 illustrates an RIQ for the ophthalmic lens of FIG. 20. As illustrated, the lens design results in two RIQ peaks—one as a result of the clear center 2101 and the second one 2102 as a result of the rings 2101.

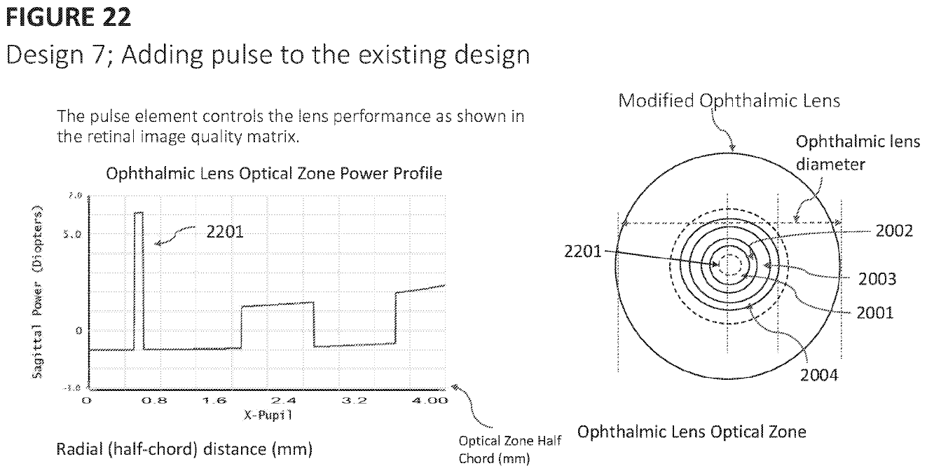

FIG. 22 illustrates an exemplary embodiment of an ophthalmic lens incorporating an exemplary optical design in accordance with some embodiments described herein. As illustrated, the ophthalmic lens isa contact lens and may comprise a base power profile similar to the profile illustrated in FIG. 20 and a single pulse 2201 within a half-chord distance of 4 mm, a width of about 0.1 mm and a power of about +6 D. As illustrated, in some embodiments, the base power profile may not be a single vision lens. For example the lens may be a bifocal lens, a dual focus lens, a trifocal lens, a multifocal lens, a zonal simultaneous vision multifocal lens, a progressive aspheric multifocal simultaneous vision lens, a toric lens, a multifocal toric lens, a myopia control lens and/or any other category including refractive, non-refractive, light scattering, light wavelength filtering, phase modulating, light amplitude modulating, metalens etc.

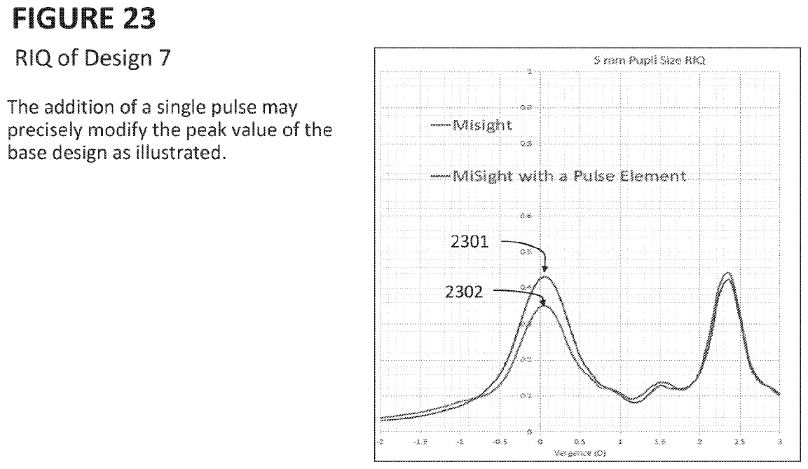

FIG. 23 illustrates an exemplary embodiment of an RIQ for the ophthalmic lens of FIG. 22. As illustrated, the lens design shows a modified (e.g., improved) RIQ performance relative to the design in FIG. 20. The peak RIQ in this example is less than about 0.4 (e.g., about 0.35). As illustrated, the addition of the pulse to the previously described base lens power results in a change (e.g., decrease) in the peak RIQ 2301 by about 10% to a peak 2302. In some embodiments, such a decrease may be desirable and may be used to alter the RIQ of a lens profile. For example, the precision of a single pulse may make a small but significant change to the RIQ at a targeted vergence without substantially changing the remainder of the RIQ plot.

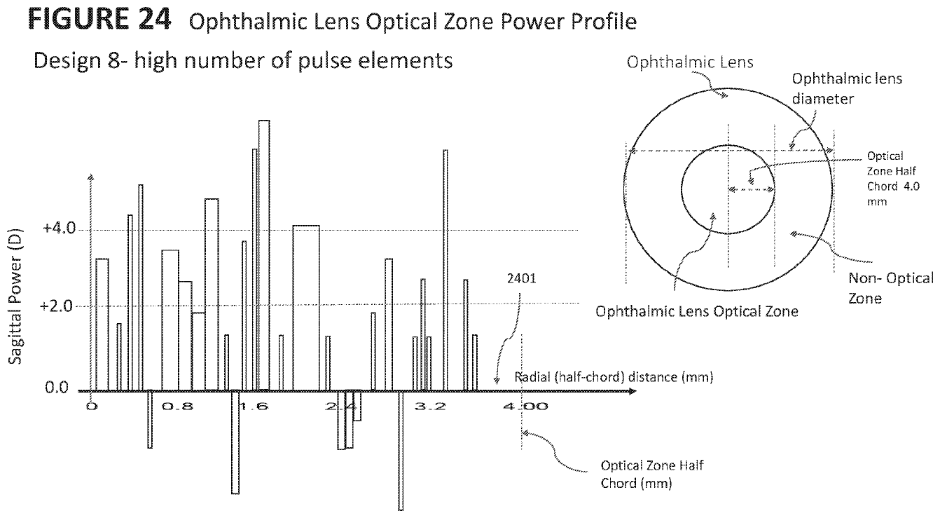

FIG. 24 illustrates an exemplary embodiment of an ophthalmic lens incorporating an exemplary optical design in accordance with some embodiments described herein. As illustrated, this embodiment is intended to illustrate an ophthalmic lens design e.g., a contact lens with a high number of pulses with varying parameters. This embodiment illustrates a design with a plurality of pulses e.g., 30 designed across the base power profile 2401 of the lens. The pulses include single pulses and conjoined pulses and may be relatively more negative pulses and relatively more positive pulses than the base power profile. The pulses also may include forms of a square pulse, where the power (height), the width and location of each pulse may be different. This example also illustrates a level of optical control that may be possible with pulse power profiles. As illustrated, every pulse of the plurality of pulses may change independently and be utilized to construct a desirable wavefront shape. In some embodiments, including multiple pulses (e.g., 30 pulses) provides a finer and/or more precise control of the design in comparison with conventional designs where there are no individual optical elements that can change independently. In other words, in some embodiments, a higher resolution of power profile control (e.g., 30 pulses) may provide improved image quality for the eye across different vergences and vision scenarios. This example also highlights the level of optical control embodiments described herein may provide because every pulse of the multiple (e.g., 30) pulses can change independently and contribute to the construction of the wavefront shape. In some embodiments, a plurality of pulses may provide greater control of image quality e.g., RIQ and enable more functionality to be included in a lens to correct and/or treat at least one condition of the eye (e.g., presbyopia, myopia, hyperopia, astigmatism, binocular vision disorders, night vision and/or visual fatigue syndrome).

In some embodiments, the resolution of optical design may only be limited by manufacturing and/or metrology.

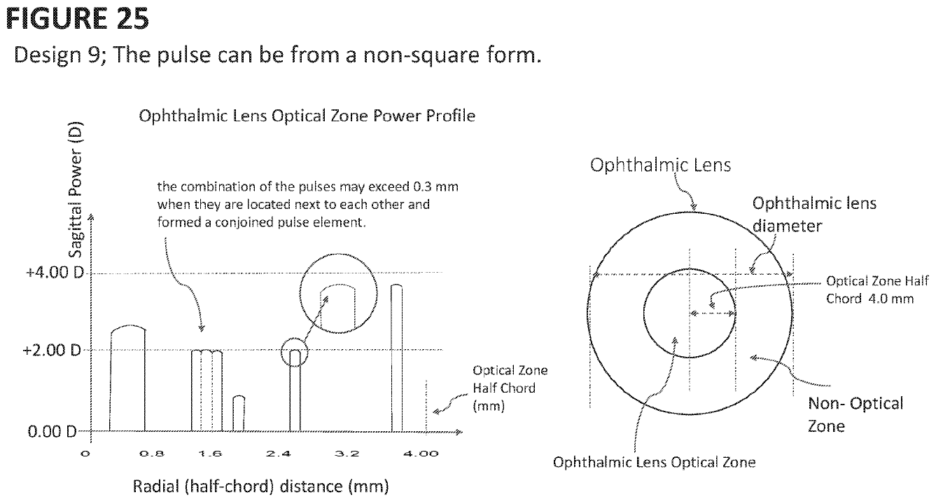

FIG. 25 illustrates an exemplary embodiment of an ophthalmic lens incorporating an exemplary optical design in accordance with some embodiments described herein. As illustrated in FIG. 25, pulses can have a variety of non-squared shapes and, in some embodiments, may be conjoined with other pulses.

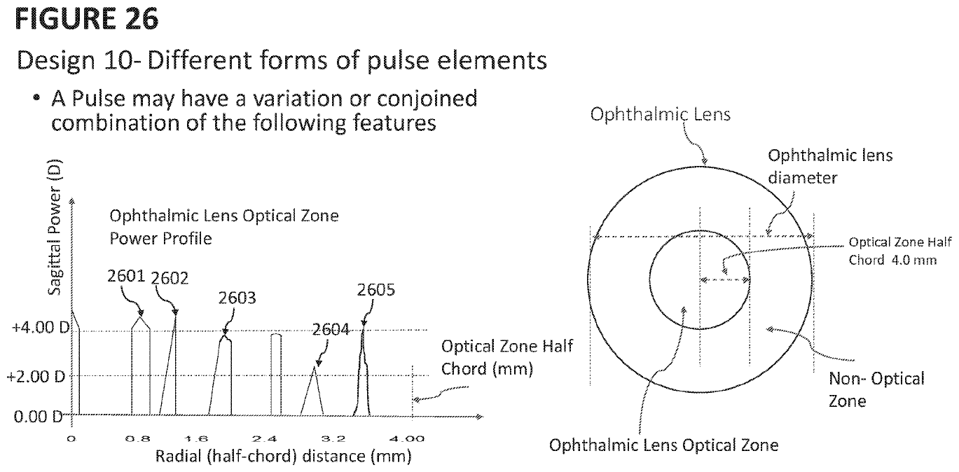

FIG. 26 illustrates an exemplary embodiment of an ophthalmic lens incorporating an exemplary optical design in accordance with some embodiments described herein. As illustrated in FIG. 26, pulses can have a variety of shapes and, in some embodiments, may be conjoined with other pulses. For example, in some embodiments, the pulses may be parallel sided or not have parallel sides (e.g. have diverging or converging sides) and the tops or peaks of pulses may be planar or non-planar. In some embodiments, the pulses may not be equal in width at the bottom of the pulse or may be irregular in shape or be aberrated or be described by a mathematical function. Pulses may generally form a concentric annular ring but may also not be concentric or annular and may form an incomplete ring or spiral or a number of partial sectors of a ring spaced apart or conjoined at least for a portion of the pulse. The pulse may be rotationally symmetrical or not symmetrical (e.g., any pulse parameter may be varied in a rotational direction or in a radial direction). In some embodiments, pulse parameters may be the same or constant or may vary and not be constant in any combination of a rotational, radial or angular direction or location to provide a finer control of the power profile and the resulting image quality.

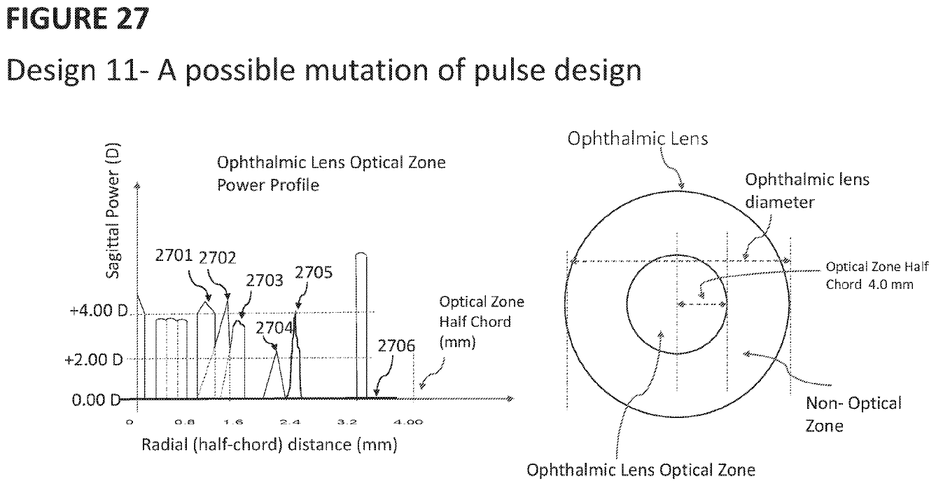

FIG. 27 illustrates an exemplary embodiment of an ophthalmic lens incorporating an exemplary optical design in accordance with some embodiments described herein. As illustrated in FIG. 27, pulses can have a variety of shapes and, in some embodiments, may be conjoined with other pulses. FIG. 27 illustrates examples of an ophthalmic lens e.g., a contact lens where the power profile includes pulses 2701, 2702 and 2703 that overlap with each other (compare with original shapes FIG. 26 as 2601, 2602 and 2603). The power profile also includes pulses 2704 and 2705 that are conjoined pulses from the individual pulses shown in FIG. 26 (e.g., pulses 2604 and 2605).

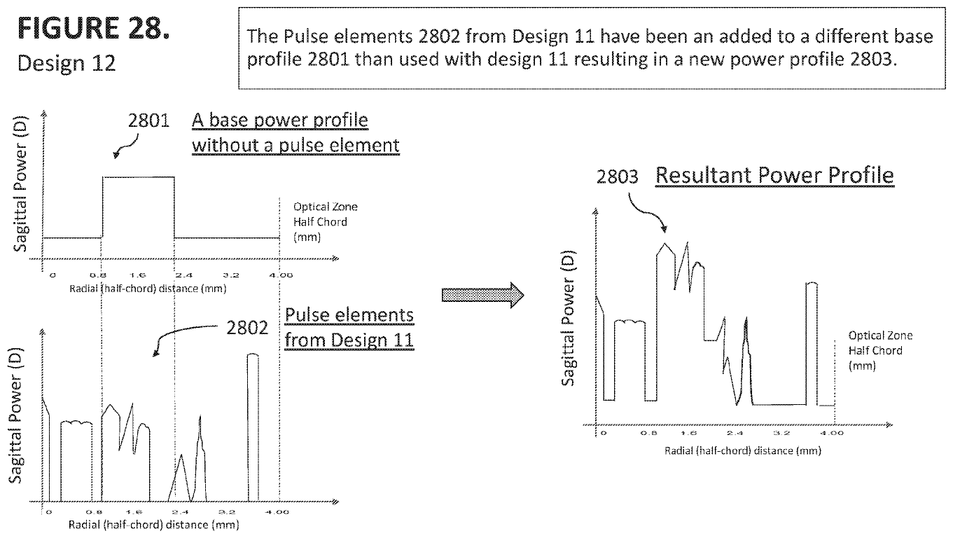

FIG. 28 illustrates an exemplary embodiment of an ophthalmic lens incorporating an exemplary optical design in accordance with some embodiments described herein. As illustrated in FIG. 28, the one or more pulses may be added to any power profile e.g., 2801 to create a power profile comprising a base power profile with one or more pulses (e.g., 2802 and 2803). In the embodiments illustrated in FIG. 28, the base power profile is changed from the FIG. 27 where the pulses are located on a flat base power profile 2706. In FIG. 28, the base power profile is changed to a different base profile 2801. The pulses 2802 (the same plurality of pulses as illustrated in FIG. 27) are then added to the new base profile 2801 to obtain the resultant power profile shown in 2803.

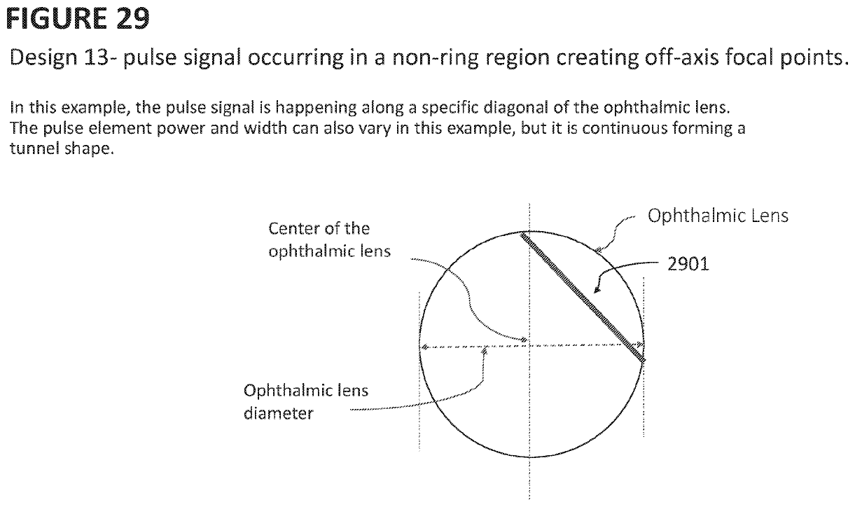

FIG. 29 illustrates an exemplary embodiment of an ophthalmic lens incorporating an exemplary optical design in accordance with some embodiments described herein. As illustrated, in some embodiments, the pulse 2901 may be created on a portion of the lens in a non-radial direction (e.g., so as not to create a concentric and/or symmetrical pulse design. In some embodiments, the at least one pulse may be astigmatic and/or not-astigmatic.

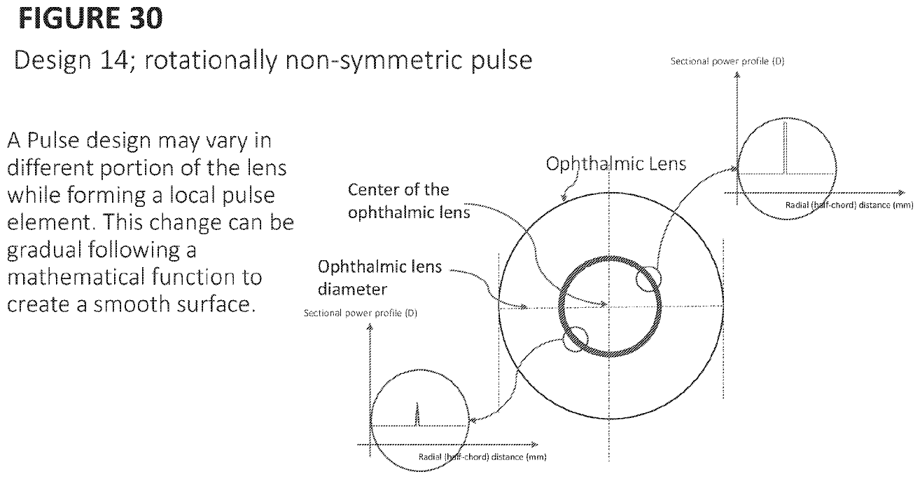

FIG. 30 illustrates an exemplary embodiment of an ophthalmic lens incorporating an exemplary optical design in accordance with some embodiments described herein. As illustrated, in some embodiments, the pulse may not be rotationally symmetric. In some embodiments, the pulse may vary across a particular dimension of the pulse. For example, in some embodiments, any combination of one or more of a width, height, spacing, distribution, and/or shape may vary.

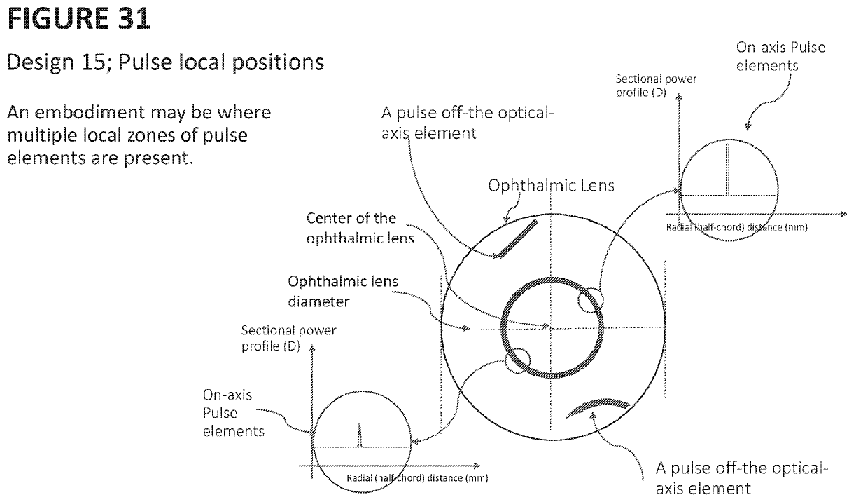

FIG. 31 illustrates an exemplary embodiment of an ophthalmic lens incorporating an exemplary optical design in accordance with some embodiments described herein. As illustrated, in some embodiments, the ophthalmic lens may comprise zones, regions and/or areas of single and/or multiple pulses.

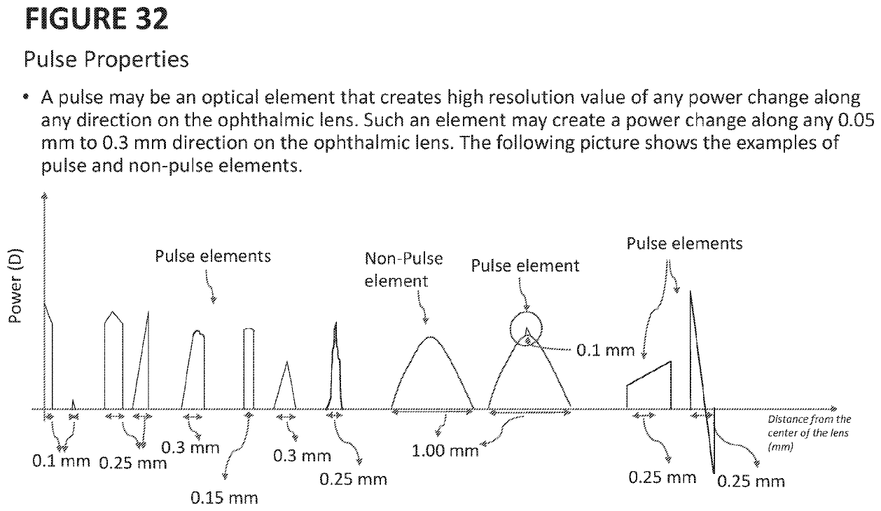

FIG. 32 illustrates exemplary properties of pulses that may be modified to obtain a desirable profile and/or RIQ modification (e.g., reduction) in accordance with some embodiments described herein. As illustrated, a pulse may be an optical element that creates high resolution value (e.g., a change in power over a small area) of any power change along any direction on the ophthalmic lens. Such an element may create a power change along any 0.05 mm to 0.3 mm direction (e.g., 0.25, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, and/or 3.5 mm) on the ophthalmic lens. The pulses may have particular widths as measured at the base profile, half-chord distances, powers/heights relative to the base profile.

### A Examples

A1. An ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile.
A2. The ophthalmic lens of any of the A examples, wherein the at least one pulse may be located on a front surface and/or a back surface of the ophthalmic lens.
A3. The ophthalmic lens of any of the A examples, wherein the at least one pulse may be formed in the matrix of the ophthalmic lens (e.g., between the front and back surfaces).
A4. The ophthalmic lens of any of the A examples, wherein the at least one pulse may be formed on a layer, a film, a lens precursor (e.g., a mold tool), a casting mold, a lens blank and/or formed on a fully finished lens as a post processing step.
A5. The ophthalmic lens of any of the A examples, wherein the at least one pulse may be formed by a material additive process (e.g., printing, stamping, dying, 3D printing, and/or a lithographic process).
A6. The ophthalmic lens of any of the A examples, wherein the at least one pulse may be formed by a material subtractive process (e.g., lasers, light energy, CO2 or UV, etching, thermal, and/or a chemical process).
A7. The ophthalmic lens of any of the A examples, wherein the at least one pulse may be formed by a material modification process (e.g., thermal, chemical changing the shape, refractive index and/or a chemical composition modification process).
A8. The ophthalmic lens of any of the A examples, wherein the at least one pulse may have a width of less than 0.3 mm.
A9. The ophthalmic lens of any of the A examples, wherein the at least one pulse may have a width of about 0.05 mm.
A10. The ophthalmic lens of any of the A examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
A11. The ophthalmic lens of any of the A examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
A12. The ophthalmic lens of any of the A examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
A13. The ophthalmic lens of any of the A examples, wherein the at least one pulse may have a shape selected from any combination of one or more of a square, conjoined of different features, triangles, rounded top, curved tops, slanted, oblique sides, and/or transition on the sides.
A14. The ophthalmic lens of any of the A examples, wherein the sides of a pulse may be parallel to each other (e.g., substantially parallel) or not parallel to each other.
A15. The ophthalmic lens of any of the A examples, wherein the tops of the pulses may be at least one of a planar, not planar, curved, monotonic, non-monotonic, periodic, and/or aperiodic profile (e.g., power profile).
A16. The ophthalmic lens of any of the A examples, wherein the at least one pulse may be a rapid, narrowband change in power.
A17. The ophthalmic lens of any of the A examples, wherein the at least one pulse may comprise a change in power comprising an increasing power component, a peak power component, and a decreasing power component relative to the base power profile.
A18. The ophthalmic lens of any of the A examples, wherein the sides of one of the at least one pulse may be connected to the base surface and a peak (or top) of the pulse may not be connected to any other feature on the lens.
A19. The ophthalmic lens of any of the A examples, wherein a first derivative of the pulse optical/power profile may be discontinuous at a boundary between the base power profile and the at least one pulse.
A20. The ophthalmic lens of any of the A examples, wherein the at least one pulse may include a discontinuity in the first derivative of the power profile in a least one location of the at least one pulse.
A21. The ophthalmic lens of any of the A examples, wherein the pulse may be a narrow width feature having a predefined width, power and/or position selected to change (e.g., reduce) at least one of the variability and magnitude of an RIQ associated with the base power profile.
A22. The ophthalmic lens of any of the A examples, wherein the change in RIQ may be at any portion of the RIQ plot.
A23. The ophthalmic lens of any of the A examples, wherein the change in RIQ may be at a particular vergence.
A24. The ophthalmic lens of any of the A examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
A25. The ophthalmic lens of any of the A examples, wherein the at least one pulse may be a positive powered pulse relative to the base power (e.g., the base power of the lens at the start of the pulse).
A26. The ophthalmic lens of any of the A examples, wherein the at least one pulse may be a negative powered pulse relative to the base power (e.g., the base power of the lens at the start of the pulse).
A27. The ophthalmic lens of any of the A examples, wherein the at least one pulse (e.g., a relatively positive, and/or negative pulse) may be configured to influence an extended depth of focus (EDOF).
A28. The ophthalmic lens of any of the A examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
A29. The ophthalmic lens of any of the A examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
A30. The ophthalmic lens of any of the A examples, wherein the at least one pulse may be a relatively positive powered pulse and a relatively negative powered pulse.
A31. The ophthalmic lens of any of the A examples, wherein the ophthalmic lens may comprise a substantially equal number (e.g., 1, 2, 3, 4, 5, 6, 7, 8, 9, or 10) of relatively positive powered pulses and a relatively negative powered pulses.
A32. The ophthalmic lens of any of the A examples, wherein the one or more pulses may have a power to correct or contribute to one or more of a distance, intermediate and/or a near power (e.g., the one or more pulses may provide a depth of focus).
A33. The ophthalmic lens of any of the A examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
A34. The ophthalmic lens of any of the A examples, wherein the one or more pulses may not correspond to one or more of a distance, intermediate and/or near power.
A35. The ophthalmic lens of any of the A examples, wherein the one or more pulses may be conjoined pulses.
A36. The ophthalmic lens of any of the A examples, wherein the one or more pulses may be a pair of conjoined pulses configured to form a ring, a portion or ring, or a plurality of portions of a ring.
A37. The ophthalmic lens of any of the A examples, wherein the one or more pulses may be a plurality of pairs of conjoined pulses configured to form a plurality of rings.
A38. The ophthalmic lens of any of the A examples, wherein the at least one pulse may be configured to modify (e.g., increase or decrease) the RIQ for myopia control.
A39. The ophthalmic lens of any of the A examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
A40. The ophthalmic lens of any of the A examples, wherein the at least one pulse may include a pulse located in the center of the lens.
A41. The ophthalmic lens of any of the A examples, wherein the at least one pulse may be created on a portion of the lens in a non-radial direction (e.g., to create an asymmetrical, non-annular, and/or non-concentric pulse).
A42. The ophthalmic lens of any of the A examples, wherein the at least one pulse may vary across a particular dimension of the pulse (e.g., any combination of one or more of a width, height, and/or shape varies in a particular dimension and/or direction).
A43. The ophthalmic lens of any of the A examples, wherein the shape of the pulse may include parallel or non-parallel side and a planar or non-planar top.
A44. The ophthalmic lens of any of the A examples, wherein the ophthalmic lens may be manufactured using at least one of lathing, molding, spinning, printing, etching, sputtering
A45. The ophthalmic lens of any of the A examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, 2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
A46. The ophthalmic lens of any of the A examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, +3.1D+3.2D, and/or +3.25D)), and wherein an RIQ area of the one or more independent peaks is about 0.16 Units*Diopters (e.g., 0.13, 0.14, 0.15, 0.16, 0.17, 0.18 or 0.19) or less.
A47. The ophthalmic lens of any of the A examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, ±3.1D, +3.2D, and/or +3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
A48. The ophthalmic lens of any of the A examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
A49. The ophthalmic lens of any of the A examples, wherein the at least one pulse may comprise a plurality of narrow and/or annular concentric optical zones located on at least one of the front surface and/or the back surface and/or within the matrix of the ophthalmic lens and formed by line curvatures.
A50. The ophthalmic lens of any of the A examples, wherein the ophthalmic lens may be one of a contact lens, an intraocular lens, and/or a spectacle lens.

### B Examples

B1. An ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile, wherein the at least one pulse is selected to create a power profile with much greater precision than possible with other lens design techniques by using the one or more pulses to create an optical design are configured to provide a higher resolution specification of a power profile.
B2. The ophthalmic lens of any of the B examples, wherein the at least one pulse may be located on a front surface and/or a back surface of the ophthalmic lens.
B3. The ophthalmic lens of any of the B examples, wherein the at least one pulse may be formed in the matrix of the ophthalmic lens (e.g., between the front and back surfaces).
B4. The ophthalmic lens of any of the B examples, wherein the at least one pulse may be formed on a layer, a film, a lens precursor (e.g., a mold tool), a casting mold, a lens blank and/or formed on a fully finished lens as a post processing step.
B5. The ophthalmic lens of any of the B examples, wherein the at least one pulse may be formed by a material additive process (e.g., printing, stamping, dying, 3D printing, and/or a lithographic process).
B6. The ophthalmic lens of any of the B examples, wherein the at least one pulse may be formed by a material subtractive process (e.g., lasers, light energy, CO2 or UV, etching, thermal, and/or a chemical process).
B7. The ophthalmic lens of any of the B examples, wherein the at least one pulse may be formed by a material modification process (e.g., thermal, chemical changing the shape, refractive index and/or a chemical composition modification process).
B8. The ophthalmic lens of any of the B examples, wherein the at least one pulse may have a width of less than 0.3 mm.
B9. The ophthalmic lens of any of the B examples, wherein the at least one pulse may have a width of about 0.05 mm.
B10. The ophthalmic lens of any of the B examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
B11. The ophthalmic lens of any of the B examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
B12. The ophthalmic lens of any of the B examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
B13. The ophthalmic lens of any of the B examples, wherein the at least one pulse may have a shape selected from any combination of one or more of a square, conjoined of different features, triangles, rounded top, curved tops, slanted, oblique sides, and/or transition on the sides.
B14. The ophthalmic lens of any of the B examples, wherein the sides of a pulse may be parallel to each other (e.g., substantially parallel) or not parallel to each other.
B15. The ophthalmic lens of any of the B examples, wherein the tops of the pulses may be at least one of a planar, not planar, curved, monotonic, non-monotonic, periodic, and/or aperiodic profile (e.g., power profile).
B16. The ophthalmic lens of any of the B examples, wherein the at least one pulse may be a rapid, narrowband change in power.
B17. The ophthalmic lens of any of the B examples, wherein the at least one pulse may comprise a change in power comprising an increasing power component, a peak power component, and a decreasing power component relative to the base power profile.
B18. The ophthalmic lens of any of the B examples, wherein the sides of one of the at least one pulse may be connected to the base surface and a peak (or top) of the pulse may not be connected to any other feature on the lens.
B19. The ophthalmic lens of any of the B examples, wherein a first derivative of the pulse optical/power profile may be discontinuous at a boundary between the base power profile and the at least one pulse.
B20. The ophthalmic lens of any of the B examples, wherein the at least one pulse may include a discontinuity in the first derivative of the power profile in a least one location of the at least one pulse.
B21. The ophthalmic lens of any of the B examples, wherein the pulse may be a narrow width feature having a predefined width, power and/or position selected to change (e.g., reduce) at least one of the variability and magnitude of an RIQ associated with the base power profile.
B22. The ophthalmic lens of any of the B examples, wherein the change in RIQ may be at any portion of the RIQ plot.
B23. The ophthalmic lens of any of the B examples, wherein the change in RIQ may be at a particular vergence.
B24. The ophthalmic lens of any of the B examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
B25. The ophthalmic lens of any of the B examples, wherein the at least one pulse may be a positive powered pulse relative to the base power (e.g., the base power of the lens at the start of the pulse).
B26. The ophthalmic lens of any of the B examples, wherein the at least one pulse may be a negative powered pulse relative to the base power (e.g., the base power of the lens at the start of the pulse).
B27. The ophthalmic lens of any of the B examples, wherein the at least one pulse (e.g., a relatively positive, and/or negative pulse) may be configured to influence an extended depth of focus (EDOF).
B28. The ophthalmic lens of any of the B examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
B29. The ophthalmic lens of any of the B examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
B30. The ophthalmic lens of any of the B examples, wherein the at least one pulse may be a relatively positive powered pulse and a relatively negative powered pulse.
B31. The ophthalmic lens of any of the B examples, wherein the ophthalmic lens may comprise a substantially equal number (e.g., 1, 2, 3, 4, 5, 6, 7, 8, 9, or 10) of relatively positive powered pulses and a relatively negative powered pulses.
B32. The ophthalmic lens of any of the B examples, wherein the one or more pulses may have a power to correct or contribute to one or more of a distance, intermediate and/or a near power (e.g., the one or more pulses may provide a depth of focus).
B33. The ophthalmic lens of any of the B examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
B34. The ophthalmic lens of any of the B examples, wherein the one or more pulses may not correspond to one or more of a distance, intermediate and/or near power.
B35. The ophthalmic lens of any of the B examples, wherein the one or more pulses may be conjoined pulses.
B36. The ophthalmic lens of any of the B examples, wherein the one or more pulses may be a pair of conjoined pulses configured to form a ring, a portion or ring, or a plurality of portions of a ring.
B37. The ophthalmic lens of any of the B examples, wherein the one or more pulses may be a plurality of pairs of conjoined pulses configured to form a plurality of rings.
B38. The ophthalmic lens of any of the B examples, wherein the at least one pulse may be configured to modify (e.g., increase or decrease) the RIQ for myopia control.
B39. The ophthalmic lens of any of the B examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
B40. The ophthalmic lens of any of the B examples, wherein the at least one pulse may include a pulse located in the center of the lens.
B41. The ophthalmic lens of any of the B examples, wherein the at least one pulse may be created on a portion of the lens in a non-radial direction (e.g., to create an asymmetrical, non-annular, and/or non-concentric pulse).
B42. The ophthalmic lens of any of the B examples, wherein the at least one pulse may vary across a particular dimension of the pulse (e.g., any combination of one or more of a width, height, and/or shape varies in a particular dimension and/or direction).
B43. The ophthalmic lens of any of the B examples, wherein the shape of the pulse may include parallel or non-parallel side and a planar or non-planar top.
B44. The ophthalmic lens of any of the B examples, wherein the ophthalmic lens may be manufactured using at least one of lathing, molding, spinning, printing, etching, sputtering
B45. The ophthalmic lens of any of the B examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
B46. The ophthalmic lens of any of the B examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, ±3.1D ±3.2D, and/or ±3.25D)), and wherein an RIQ area of the one or more independent peaks is about 0.16 Units*Diopters (e.g., 0.13, 0.14, 0.15, 0.16, 0.17, 0.18 or 0.19) or less.
B47. The ophthalmic lens of any of the B examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, ±3.1D, +3.2D, and/or +3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
B48. The ophthalmic lens of any of the B examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
B49. The ophthalmic lens of any of the B examples, wherein the at least one pulse may comprise a plurality of narrow and/or annular concentric optical zones located on at least one of the front surface and/or the back surface and/or within the matrix of the ophthalmic lens and formed by line curvatures.
B50. The ophthalmic lens of any of the B examples, wherein the ophthalmic lens may be one of a contact lens, an intraocular lens, and/or a spectacle lens.

### C Examples

C1. An ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile, wherein the one or more pulses may be introduced to a base power profile to modify (e.g., increase, decrease, eliminate or create) one or more RIQ values (e.g., peak RIQ values) at any desired vergence along an RIQ curve (e.g., for a bifocal base power profile with two RIQ peaks—one at a distance vergence and one at a near vergence—one or more pulses may be introduced that create a new RIQ peak at an intermediate vergence to create a trifocal ophthalmic lens.
C2. The ophthalmic lens of any of the C examples, wherein the design may target the redistribution of a defined light energy away from the two bifocal RIQ peaks and/or a plurality of pulses are designed to introduce multiple spaced apart RIQ peaks or a broader continuous RIQ region between the distance and near RIQ peaks of the base power to create an extended depth of focus (EDOF) while modifying (e.g. reducing) the light energy directed to the two bifocal peaks formed by the conventional pulses.
C3. The ophthalmic lens of any of the C examples, wherein the at least one pulse may be located on a front surface and/or a back surface of the ophthalmic lens.
C4. The ophthalmic lens of any of the C examples, wherein the at least one pulse may be formed in the matrix of the ophthalmic lens (e.g., between the front and back surfaces).
C5. The ophthalmic lens of any of the C examples, wherein the at least one pulse may be formed on a layer, a film, a lens precursor (e.g., a mold tool), a casting mold, a lens blank and/or formed on a fully finished lens as a post processing step.
C6. The ophthalmic lens of any of the C examples, wherein the at least one pulse may be formed by a material additive process (e.g., printing, stamping, dying, 3D printing, and/or a lithographic process).
C7. The ophthalmic lens of any of the C examples, wherein the at least one pulse may be formed by a material subtractive process (e.g., lasers, light energy, CO2 or UV, etching, thermal, and/or a chemical process).
C8. The ophthalmic lens of any of the C examples, wherein the at least one pulse may be formed by a material modification process (e.g., thermal, chemical changing the shape, refractive index and/or a chemical composition modification process).
C9. The ophthalmic lens of any of the C examples, wherein the at least one pulse may have a width of less than 0.3 mm.
C10. The ophthalmic lens of any of the C examples, wherein the at least one pulse may have a width of about 0.05 mm.
C11. The ophthalmic lens of any of the C examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
C12. The ophthalmic lens of any of the C examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
C13. The ophthalmic lens of any of the C examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
C14. The ophthalmic lens of any of the C examples, wherein the at least one pulse may have a shape selected from any combination of one or more of a square, conjoined of different features, triangles, rounded top, curved tops, slanted, oblique sides, and/or transition on the sides.
C15. The ophthalmic lens of any of the C examples, wherein the sides of a pulse may be parallel to each other (e.g., substantially parallel) or not parallel to each other.
C16. The ophthalmic lens of any of the C examples, wherein the tops of the pulses may be at least one of a planar, not planar, curved, monotonic, non-monotonic, periodic, and/or aperiodic profile (e.g., power profile).
C17. The ophthalmic lens of any of the C examples, wherein the at least one pulse may be a rapid, narrowband change in power.
C18. The ophthalmic lens of any of the C examples, wherein the at least one pulse may comprise a change in power comprising an increasing power component, a peak power component, and a decreasing power component relative to the base power profile.
C19. The ophthalmic lens of any of the C examples, wherein the sides of one of the at least one pulse may be connected to the base surface and a peak (or top) of the pulse may not be connected to any other feature on the lens.
C20. The ophthalmic lens of any of the C examples, wherein a first derivative of the pulse optical/power profile may be discontinuous at a boundary between the base power profile and the at least one pulse.
C21. The ophthalmic lens of any of the C examples, wherein the at least one pulse may include a discontinuity in the first derivative of the power profile in a least one location of the at least one pulse.
C22. The ophthalmic lens of any of the C examples, wherein the pulse may be a narrow width feature having a predefined width, power and/or position selected to change (e.g., reduce) at least one of the variability and magnitude of an RIQ associated with the base power profile.
C23. The ophthalmic lens of any of the C examples, wherein the change in RIQ may be at any portion of the RIQ plot.
C24. The ophthalmic lens of any of the C examples, wherein the change in RIQ may be at a particular vergence.
C25. The ophthalmic lens of any of the C examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
C26. The ophthalmic lens of any of the C examples, wherein the at least one pulse may be a positive powered pulse relative to the base power (e.g., the base power of the lens at the start of the pulse).
C27. The ophthalmic lens of any of the C examples, wherein the at least one pulse may be a negative powered pulse relative to the base power (e.g., the base power of the lens at the start of the pulse).
C28. The ophthalmic lens of any of the C examples, wherein the at least one pulse (e.g., a relatively positive, and/or negative pulse) may be configured to influence an extended depth of focus (EDOF).
C29. The ophthalmic lens of any of the C examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
C30. The ophthalmic lens of any of the C examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
C31. The ophthalmic lens of any of the C examples, wherein the at least one pulse may be a relatively positive powered pulse and a relatively negative powered pulse.
C32. The ophthalmic lens of any of the C examples, wherein the ophthalmic lens may comprise a substantially equal number (e.g., 1, 2, 3, 4, 5, 6, 7, 8, 9, or 10) of relatively positive powered pulses and a relatively negative powered pulses.
C33. The ophthalmic lens of any of the C examples, wherein the one or more pulses may have a power to correct or contribute to one or more of a distance, intermediate and/or a near power (e.g., the one or more pulses may provide a depth of focus).
C34. The ophthalmic lens of any of the C examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
C35. The ophthalmic lens of any of the C examples, wherein the one or more pulses may not correspond to one or more of a distance, intermediate and/or near power.
C36. The ophthalmic lens of any of the C examples, wherein the one or more pulses may be conjoined pulses.
C37. The ophthalmic lens of any of the C examples, wherein the one or more pulses may be a pair of conjoined pulses configured to form a ring, a portion or ring, or a plurality of portions of a ring.
C38. The ophthalmic lens of any of the C examples, wherein the one or more pulses may be a plurality of pairs of conjoined pulses configured to form a plurality of rings.
C39. The ophthalmic lens of any of the C examples, wherein the at least one pulse may be configured to modify (e.g., increase or decrease) the RIQ for myopia control.
C40. The ophthalmic lens of any of the C examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
C41. The ophthalmic lens of any of the C examples, wherein the at least one pulse may include a pulse located in the center of the lens.
C42. The ophthalmic lens of any of the C examples, wherein the at least one pulse may be created on a portion of the lens in a non-radial direction (e.g., to create an asymmetrical, non-annular, and/or non-concentric pulse).
C43. The ophthalmic lens of any of the C examples, wherein the at least one pulse may vary across a particular dimension of the pulse (e.g., any combination of one or more of a width, height, and/or shape varies in a particular dimension and/or direction).
C44. The ophthalmic lens of any of the C examples, wherein the shape of the pulse may include parallel or non-parallel side and a planar or non-planar top.
C45. The ophthalmic lens of any of the C examples, wherein the ophthalmic lens may be manufactured using at least one of lathing, molding, spinning, printing, etching, sputtering
C46. The ophthalmic lens of any of the C examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
C47. The ophthalmic lens of any of the C examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, ±3.1D ±3.2D, and/or ±3.25D)), and wherein an RIQ area of the one or more independent peaks is about 0.16 Units*Diopters (e.g., 0.13, 0.14, 0.15, 0.16, 0.17, 0.18 or 0.19) or less.
C48. The ophthalmic lens of any of the C examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, ±3.1D, +3.2D, and/or +3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
C49. The ophthalmic lens of any of the C examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
C50. The ophthalmic lens of any of the C examples, wherein the at least one pulse may comprise a plurality of narrow and/or annular concentric optical zones located on at least one of the front surface and/or the back surface and/or within the matrix of the ophthalmic lens and formed by line curvatures.
C51. The ophthalmic lens of any of the C examples, wherein the ophthalmic lens may be one of a contact lens, an intraocular lens, and/or a spectacle lens.

### D Examples

D1. An ophthalmic lens configured to correct and/or treat at least one condition of the eye (e.g., presbyopia, myopia, hyperopia, astigmatism, binocular vision disorders, night vision image quality, and/or visual fatigue syndrome) comprising: a base power profile; and at least one pulse selected to modify the base power profile, wherein the one or more pulses may be introduced to a base power profile (e.g. a multifocal base power profile formed without pulses) based on the simultaneous vision principle and the one or more pulses may be configured to selectively modify an RIQ at any vergence (e.g. increase, decrease, eliminate and/or create peaks, across the useful range of vergences for vision).
D2. The ophthalmic lens of any of the D examples, wherein the one or more pulses may reduce the light energy of the RIQ plot at the selected vergences to a relatively low level while maintaining it sufficiently high for good vision at other vergences (e.g., distance, intermediate, near and/or anywhere in between).
D3. The ophthalmic lens of any of the D examples, wherein the one or more pulses may be configured so that the undesired light energy is spread across a range of vergences (e.g., outside the useful range of vergences for presbyopic vision) and/or the interference from out of focus light energy on an in focus image plane is substantially reduced and/or minimized because the light energy is widely spread and of relatively low energy at the vergence vergences (e.g. a low RIQ) including within the useful range of vergences.
D4. The ophthalmic lens of any of the D examples, wherein the at least one pulse may be located on a front surface and/or a back surface of the ophthalmic lens.
D5. The ophthalmic lens of any of the D examples, wherein the at least one pulse may be formed in the matrix of the ophthalmic lens (e.g., between the front and back surfaces).
D6. The ophthalmic lens of any of the D examples, wherein the at least one pulse may be formed on a layer, a film, a lens precursor (e.g., a mold tool), a casting mold, a lens blank and/or formed on a fully finished lens as a post processing step.
D7. The ophthalmic lens of any of the D examples, wherein the at least one pulse may be formed by a material additive process (e.g., printing, stamping, dying, 3D printing, and/or a lithographic process).
D8. The ophthalmic lens of any of the D examples, wherein the at least one pulse may be formed by a material subtractive process (e.g., lasers, light energy, CO2 or UV, etching, thermal, and/or a chemical process).
D9. The ophthalmic lens of any of the D examples, wherein the at least one pulse may be formed by a material modification process (e.g., thermal, chemical changing the shape, refractive index and/or a chemical composition modification process).
D10. The ophthalmic lens of any of the D examples, wherein the at least one pulse may have a width of less than 0.3 mm.
D11. The ophthalmic lens of any of the D examples, wherein the at least one pulse may have a width of about 0.05 mm.
D12. The ophthalmic lens of any of the D examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
D13. The ophthalmic lens of any of the D examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
D14. The ophthalmic lens of any of the D examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
D15. The ophthalmic lens of any of the D examples, wherein the at least one pulse may have a shape selected from any combination of one or more of a square, conjoined of different features, triangles, rounded top, curved tops, slanted, oblique sides, and/or transition on the sides.
D16. The ophthalmic lens of any of the D examples, wherein the sides of a pulse may be parallel to each other (e.g., substantially parallel) or not parallel to each other.
D17. The ophthalmic lens of any of the D examples, wherein the tops of the pulses may be at least one of a planar, not planar, curved, monotonic, non-monotonic, periodic, and/or aperiodic profile (e.g., power profile).
D18. The ophthalmic lens of any of the D examples, wherein the at least one pulse may be a rapid, narrowband change in power.
D19. The ophthalmic lens of any of the D examples, wherein the at least one pulse may comprise a change in power comprising an increasing power component, a peak power component, and a decreasing power component relative to the base power profile.
D20. The ophthalmic lens of any of the D examples, wherein the sides of one of the at least one pulse may be connected to the base surface and a peak (or top) of the pulse may not be connected to any other feature on the lens.
D21. The ophthalmic lens of any of the D examples, wherein a first derivative of the pulse optical/power profile may be discontinuous at a boundary between the base power profile and the at least one pulse.
D22. The ophthalmic lens of any of the D examples, wherein the at least one pulse may include a discontinuity in the first derivative of the power profile in a least one location of the at least one pulse.
D23. The ophthalmic lens of any of the D examples, wherein the pulse may be a narrow width feature having a predefined width, power and/or position selected to change (e.g., reduce) at least one of the variability and magnitude of an RIQ associated with the base power profile.
D24. The ophthalmic lens of any of the D examples, wherein the change in RIQ may be at any portion of the RIQ plot.
D25. The ophthalmic lens of any of the D examples, wherein the change in RIQ may be at a particular vergence.
D26. The ophthalmic lens of any of the D examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
D27. The ophthalmic lens of any of the D examples, wherein the at least one pulse may be a positive powered pulse relative to the base power (e.g., the base power of the lens at the start of the pulse).
D28. The ophthalmic lens of any of the D examples, wherein the at least one pulse may be a negative powered pulse relative to the base power (e.g., the base power of the lens at the start of the pulse).
D29. The ophthalmic lens of any of the D examples, wherein the at least one pulse (e.g., a relatively positive, and/or negative pulse) may be configured to influence an extended depth of focus (EDOF).
D30. The ophthalmic lens of any of the D examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
D31. The ophthalmic lens of any of the D examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
D32. The ophthalmic lens of any of the D examples, wherein the at least one pulse may be a relatively positive powered pulse and a relatively negative powered pulse.
D33. The ophthalmic lens of any of the D examples, wherein the ophthalmic lens may comprise a substantially equal number (e.g., 1, 2, 3, 4, 5, 6, 7, 8, 9, or 10) of relatively positive powered pulses and a relatively negative powered pulses.
D34. The ophthalmic lens of any of the D examples, wherein the one or more pulses may have a power to correct or contribute to one or more of a distance, intermediate and/or a near power (e.g., the one or more pulses may provide a depth of focus).
D35. The ophthalmic lens of any of the D examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
D36. The ophthalmic lens of any of the D examples, wherein the one or more pulses may not correspond to one or more of a distance, intermediate and/or near power.
D37. The ophthalmic lens of any of the D examples, wherein the one or more pulses may be conjoined pulses.
D38. The ophthalmic lens of any of the D examples, wherein the one or more pulses may be a pair of conjoined pulses configured to form a ring, a portion or ring, or a plurality of portions of a ring.
D39. The ophthalmic lens of any of the D examples, wherein the one or more pulses may be a plurality of pairs of conjoined pulses configured to form a plurality of rings.
D40. The ophthalmic lens of any of the D examples, wherein the at least one pulse may be configured to modify (e.g., increase or decrease) the RIQ for myopia control.
D41. The ophthalmic lens of any of the D examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
D42. The ophthalmic lens of any of the D examples, wherein the at least one pulse may include a pulse located in the center of the lens.
D43. The ophthalmic lens of any of the D examples, wherein the at least one pulse may be created on a portion of the lens in a non-radial direction (e.g., to create an asymmetrical, non-annular, and/or non-concentric pulse).
D44. The ophthalmic lens of any of the D examples, wherein the at least one pulse may vary across a particular dimension of the pulse (e.g., any combination of one or more of a width, height, and/or shape varies in a particular dimension and/or direction).
D45. The ophthalmic lens of any of the D examples, wherein the shape of the pulse may include parallel or non-parallel side and a planar or non-planar top.
D46. The ophthalmic lens of any of the D examples, wherein the ophthalmic lens may be manufactured using at least one of lathing, molding, spinning, printing, etching, sputtering
D47. The ophthalmic lens of any of the D examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
D48. The ophthalmic lens of any of the D examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, ±3.1D ±3.2D, and/or ±3.25D)), and wherein an RIQ area of the one or more independent peaks is about 0.16 Units*Diopters (e.g., 0.13, 0.14, 0.15, 0.16, 0.17, 0.18 or 0.19) or less.
D49. The ophthalmic lens of any of the D examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, ±3.1D, ±3.2D, and/or ±3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
D50. The ophthalmic lens of any of the D examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
D51. The ophthalmic lens of any of the D examples, wherein the at least one pulse may comprise a plurality of narrow and/or annular concentric optical zones located on at least one of the front surface and/or the back surface and/or within the matrix of the ophthalmic lens and formed by line curvatures.
D52. The ophthalmic lens of any of the D examples, wherein the ophthalmic lens may be one of a contact lens, an intraocular lens, and/or a spectacle lens.

### E Examples

E1. An ophthalmic lens configured to correct and/or treat at least one condition of the eye (e.g., presbyopia, myopia, hyperopia, astigmatism, binocular vision disorders and/or visual fatigue syndrome) comprising: a base power profile; and at least one pulse (e.g., a plurality of pulses) selected to modify the base power profile and to form one or more on-axis focal points in front of, on, and/or behind a retinal image plane and/or control a focal point energy level at one or more image planes (e.g., modify, eliminate, reduce, and/or increase a focal point energy at a particular image plane, including reducing a peak focal point energy).
E2. The ophthalmic lens of any of the E examples, wherein the at least one pulse may be located on a front surface and/or a back surface of the ophthalmic lens.
E3. The ophthalmic lens of any of the E examples, wherein the at least one pulse may be formed in the matrix of the ophthalmic lens (e.g., between the front and back surfaces).
E4. The ophthalmic lens of any of the E examples, wherein the at least one pulse may be formed on a layer, a film, a lens precursor (e.g., a mold tool), a casting mold, a lens blank and/or formed on a fully finished lens as a post processing step.
E5. The ophthalmic lens of any of the E examples, wherein the at least one pulse may be formed by a material additive process (e.g., printing, stamping, dying, 3D printing, and/or a lithographic process).
E6. The ophthalmic lens of any of the E examples, wherein the at least one pulse may be formed by a material subtractive process (e.g., lasers, light energy, CO2 or UV, etching, thermal, and/or a chemical process).
E7. The ophthalmic lens of any of the E examples, wherein the at least one pulse may be formed by a material modification process (e.g., thermal, chemical changing the shape, refractive index and/or a chemical composition modification process).
E8. The ophthalmic lens of any of the E examples, wherein the at least one pulse may have a width of less than 0.3 mm.
E9. The ophthalmic lens of any of the E examples, wherein the at least one pulse may have a width of about 0.05 mm.
E10. The ophthalmic lens of any of the E examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
E11. The ophthalmic lens of any of the E examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
E12. The ophthalmic lens of any of the E examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
E13. The ophthalmic lens of any of the E examples, wherein the at least one pulse may have a shape selected from any combination of one or more of a square, conjoined of different features, triangles, rounded top, curved tops, slanted, oblique sides, and/or transition on the sides.
E14. The ophthalmic lens of any of the E examples, wherein the sides of a pulse may be parallel to each other (e.g., substantially parallel) or not parallel to each other.
E15. The ophthalmic lens of any of the E examples, wherein the tops of the pulses may be at least one of a planar, not planar, curved, monotonic, non-monotonic, periodic, and/or aperiodic profile (e.g., power profile).
E16. The ophthalmic lens of any of the E examples, wherein the at least one pulse may be a rapid, narrowband change in power.
E17. The ophthalmic lens of any of the E examples, wherein the at least one pulse may comprise a change in power comprising an increasing power component, a peak power component, and a decreasing power component relative to the base power profile.
E18. The ophthalmic lens of any of the E examples, wherein the sides of one of the at least one pulse may be connected to the base surface and a peak (or top) of the pulse may not be connected to any other feature on the lens.
E19. The ophthalmic lens of any of the E examples, wherein a first derivative of the pulse optical/power profile may be discontinuous at a boundary between the base power profile and the at least one pulse.
E20. The ophthalmic lens of any of the E examples, wherein the at least one pulse may include a discontinuity in the first derivative of the power profile in a least one location of the at least one pulse.
E21. The ophthalmic lens of any of the E examples, wherein the pulse may be a narrow width feature having a predefined width, power and/or position selected to change (e.g., reduce) at least one of the variability and magnitude of an RIQ associated with the base power profile.
E22. The ophthalmic lens of any of the E examples, wherein the change in RIQ may be at any portion of the RIQ plot.
E23. The ophthalmic lens of any of the E examples, wherein the change in RIQ may be at a particular vergence.
E24. The ophthalmic lens of any of the E examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
E25. The ophthalmic lens of any of the E examples, wherein the at least one pulse may be a positive powered pulse relative to the base power (e.g., the base power of the lens at the start of the pulse).
E26. The ophthalmic lens of any of the E examples, wherein the at least one pulse may be a negative powered pulse relative to the base power (e.g., the base power of the lens at the start of the pulse).
E27. The ophthalmic lens of any of the E examples, wherein the at least one pulse (e.g., a relatively positive, and/or negative pulse) may be configured to influence an extended depth of focus (EDOF).
E28. The ophthalmic lens of any of the E examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
E29. The ophthalmic lens of any of the E examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
E30. The ophthalmic lens of any of the E examples, wherein the at least one pulse may be a relatively positive powered pulse and a relatively negative powered pulse.
E31. The ophthalmic lens of any of the E examples, wherein the ophthalmic lens may comprise a substantially equal number (e.g., 1, 2, 3, 4, 5, 6, 7, 8, 9, or 10) of relatively positive powered pulses and a relatively negative powered pulses.
E32. The ophthalmic lens of any of the E examples, wherein the one or more pulses may have a power to correct or contribute to one or more of a distance, intermediate and/or a near power (e.g., the one or more pulses may provide a depth of focus).
E33. The ophthalmic lens of any of the E examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
E34. The ophthalmic lens of any of the E examples, wherein the one or more pulses may not correspond to one or more of a distance, intermediate and/or near power.
E35. The ophthalmic lens of any of the E examples, wherein the one or more pulses may be conjoined pulses.
E36. The ophthalmic lens of any of the E examples, wherein the one or more pulses may be a pair of conjoined pulses configured to form a ring, a portion or ring, or a plurality of portions of a ring.
E37. The ophthalmic lens of any of the E examples, wherein the one or more pulses may be a plurality of pairs of conjoined pulses configured to form a plurality of rings.
E38. The ophthalmic lens of any of the E examples, wherein the at least one pulse may be configured to modify (e.g., increase or decrease) the RIQ for myopia control.
E39. The ophthalmic lens of any of the E examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
E40. The ophthalmic lens of any of the E examples, wherein the at least one pulse may include a pulse located in the center of the lens.
E41. The ophthalmic lens of any of the E examples, wherein the at least one pulse may be created on a portion of the lens in a non-radial direction (e.g., to create an asymmetrical, non-annular, and/or non-concentric pulse).
E42. The ophthalmic lens of any of the E examples, wherein the at least one pulse may vary across a particular dimension of the pulse (e.g., any combination of one or more of a width, height, and/or shape varies in a particular dimension and/or direction).
E43. The ophthalmic lens of any of the E examples, wherein the shape of the pulse may include parallel or non-parallel side and a planar or non-planar top.
E44. The ophthalmic lens of any of the E examples, wherein the ophthalmic lens may be manufactured using at least one of lathing, molding, spinning, printing, etching, sputtering
E45. The ophthalmic lens of any of the E examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
E46. The ophthalmic lens of any of the E examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, ±3.1D ±3.2D, and/or ±3.25D)), and wherein an RIQ area of the one or more independent peaks is about 0.16 Units*Diopters (e.g., 0.13, 0.14, 0.15, 0.16, 0.17, 0.18 or 0.19) or less.
E47. The ophthalmic lens of any of the E examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, ±3.1D, +3.2D, and/or +3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
E48. The ophthalmic lens of any of the E examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
E49. The ophthalmic lens of any of the E examples, wherein the at least one pulse may comprise a plurality of narrow and/or annular concentric optical zones located on at least one of the front surface and/or the back surface and/or within the matrix of the ophthalmic lens and formed by line curvatures.
E50. The ophthalmic lens of any of the E examples, wherein the ophthalmic lens may be one of a contact lens, an intraocular lens, and/or a spectacle lens.

### F Examples

F1. A method for changing an image quality of an ophthalmic lens design comprising: identifying a base power profile of the ophthalmic lens design; obtaining an image quality metric (e.g., an RIQ) of the base power profile of ophthalmic lens design; and adding one or more pulses to the base power profile of the ophthalmic lens design to generate a modified power profile that changes the image quality metric (e.g., reduces the RIQ and/or a peak RIQ) of the ophthalmic lens design.
F2. The method of any of the F examples, wherein the one or more pulses may modify the RIQ at one or more targeted vergences.
F3. The method of any of the F examples, wherein the modification may be within the visually usable range of vergences used for vision (e.g., +/−3D).
F4. The method of any of the F examples, wherein the modification may be beyond the visually usable range to spread unwanted or unnecessary or not needed light energy and/or reduce the interference of out of focus light energy on the in focus light energy within the useful range of vergences (e.g., along a depth of focus or at any other selected vergence).
F5. The method of any of the F examples, wherein the one or more pulses may provide an improved optical design tool to redistribute (e.g., precisely redistribute) light energy to or away from an image plane.
F6. The method of any of the F examples, wherein the one or more pulses may enable the optical characteristics of an ophthalmic lens to be specified with greater (e.g., higher) resolution so that light energy from an ophthalmic lens may also be directed at a higher resolution to modify the retinal image quality and/or light energy at each focal point with greater precision.
F7. The method of any of the F examples, wherein the one or more pulses may be designed not to alter the RIQ but may be used to modify (e.g. introduce or eliminate or increase or decrease) an optical artefact or phenomenon that may be desirable or not desirable for a wearer of the ophthalmic lens (e.g. a light disturbance effect, a halo or a starburst, a ghost image, a dysphotopsia diffraction effect from a junction or a prismatic effect, a chromatic effect, a lens distortion, and/or an image jumper of swim).
F8. The method of any of the F examples, wherein the artefact may be a residual from a manufacturing process or an inherent design feature of an ophthalmic lens that may not be readily modifiable.
F9. The method of any of the F examples, wherein the at least one pulse may be located on a front surface and/or a back surface of the ophthalmic lens.
F10. The method of any of the F examples, wherein the at least one pulse may be formed in the matrix of the ophthalmic lens (e.g., between the front and back surfaces).
F11. The method of any of the F examples, wherein the at least one pulse may be formed on a layer, a film, a lens precursor (e.g., a mold tool), a casting mold, a lens blank and/or formed on a fully finished lens as a post processing step.
F12. The method of any of the F examples, wherein the at least one pulse may be formed by a material additive process (e.g., printing, stamping, dying, 3D printing, and/or a lithographic process).
F13. The method of any of the F examples, wherein the at least one pulse may be formed by a material subtractive process (e.g., lasers, light energy, CO2 or UV, etching, thermal, and/or a chemical process).
F14. The method of any of the F examples, wherein the at least one pulse may be formed by a material modification process (e.g., thermal, chemical changing the shape, refractive index and/or a chemical composition modification process).
F15. The method of any of the F examples, wherein the at least one pulse may have a width of less than 0.3 mm.
F16. The method of any of the F examples, wherein the at least one pulse may have a width of about 0.05 mm.
F17. The method of any of the F examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
F18. The method of any of the F examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
F19. The method of any of the F examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
F20. The method of any of the F examples, wherein the at least one pulse may have a shape selected from any combination of one or more of a square, conjoined of different features, triangles, rounded top, curved tops, slanted, oblique sides, and/or transition on the sides.
F21. The method of any of the F examples, wherein the sides of a pulse may be parallel to each other (e.g., substantially parallel) or not parallel to each other.
F22. The method of any of the F examples, wherein the tops of the pulses may be at least one of a planar, not planar, curved, monotonic, non-monotonic, periodic, and/or aperiodic profile (e.g., power profile).
F23. The method of any of the F examples, wherein the at least one pulse may be a rapid, narrowband change in power.
F24. The method of any of the F examples, wherein the at least one pulse may comprise a change in power comprising an increasing power component, a peak power component, and a decreasing power component relative to the base power profile.
F25. The method of any of the F examples, wherein the sides of one of the at least one pulse may be connected to the base surface and a peak (or top) of the pulse may not be connected to any other feature on the lens.
F26. The method of any of the F examples, wherein a first derivative of the pulse optical/power profile may be discontinuous at a boundary between the base power profile and the at least one pulse.
F27. The method of any of the F examples, wherein the at least one pulse may include a discontinuity in the first derivative of the power profile in a least one location of the at least one pulse.
F28. The method of any of the F examples, wherein the pulse may be a narrow width feature having a predefined width, power and/or position selected to change (e.g., reduce) at least one of the variability and magnitude of an RIQ associated with the base power profile.
F29. The method of any of the F examples, wherein the change in RIQ may be at any portion of the RIQ plot.
F30. The method of any of the F examples, wherein the change in RIQ may be at a particular vergence.
F31. The method of any of the F examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
F32. The method of any of the F examples, wherein the at least one pulse may be a positive powered pulse relative to the base power (e.g., the base power of the lens at the start of the pulse).
F33. The method of any of the F examples, wherein the at least one pulse may be a negative powered pulse relative to the base power (e.g., the base power of the lens at the start of the pulse).
F34. The method of any of the F examples, wherein the at least one pulse (e.g., a relatively positive, and/or negative pulse) may be configured to influence an extended depth of focus (EDOF).
F35. The method of any of the F examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
F36. The method of any of the F examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
F37. The method of any of the F examples, wherein the at least one pulse may be a relatively positive powered pulse and a relatively negative powered pulse.
F38. The method of any of the F examples, wherein the ophthalmic lens may comprise a substantially equal number (e.g., 1, 2, 3, 4, 5, 6, 7, 8, 9, or 10) of relatively positive powered pulses and a relatively negative powered pulses.
F39. The method of any of the F examples, wherein the one or more pulses may have a power to correct or contribute to one or more of a distance, intermediate and/or a near power (e.g., the one or more pulses may provide a depth of focus).
F40. The method of any of the F examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
F41. The method of any of the F examples, wherein the one or more pulses may not correspond to one or more of a distance, intermediate and/or near power.
F42. The method of any of the F examples, wherein the one or more pulses may be conjoined pulses.
F43. The method of any of the F examples, wherein the one or more pulses may be a pair of conjoined pulses configured to form a ring, a portion or ring, or a plurality of portions of a ring.
F44. The method of any of the F examples, wherein the one or more pulses may be a plurality of pairs of conjoined pulses configured to form a plurality of rings.
F45. The method of any of the F examples, wherein the at least one pulse may be configured to modify (e.g., increase or decrease) the RIQ for myopia control.
F46. The method of any of the F examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
F47. The method of any of the F examples, wherein the at least one pulse may include a pulse located in the center of the lens.
F48. The method of any of the F examples, wherein the at least one pulse may be created on a portion of the lens in a non-radial direction (e.g., to create an asymmetrical, non-annular, and/or non-concentric pulse).
F49. The method of any of the F examples, wherein the at least one pulse may vary across a particular dimension of the pulse (e.g., any combination of one or more of a width, height, and/or shape varies in a particular dimension and/or direction).
F50. The method of any of the F examples, wherein the shape of the pulse may include parallel or non-parallel side and a planar or non-planar top.
F51. The method of any of the F examples, wherein the ophthalmic lens may be manufactured using at least one of lathing, molding, spinning, printing, etching, sputtering
F52. The method of any of the F examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
F53. The method of any of the F examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, ±3.1D ±3.2D, and/or ±3.25D)), and wherein an RIQ area of the one or more independent peaks is about 0.16 Units*Diopters (e.g., 0.13, 0.14, 0.15, 0.16, 0.17, 0.18 or 0.19) or less.
F54. The method of any of the F examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, 3.1D, +3.2D, and/or +3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
F55. The method of any of the F examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
F56. The method of any of the F examples, wherein the at least one pulse may comprise a plurality of narrow and/or annular concentric optical zones located on at least one of the front surface and/or the back surface and/or within the matrix of the ophthalmic lens and formed by line curvatures.
F57. The method of any of the F examples, wherein the ophthalmic lens may be one of a contact lens, an intraocular lens, and/or a spectacle lens.

### G Examples

G1. An ophthalmic lens configured to correct and/or treat at least one condition of the eye (e.g., presbyopia, myopia, hyperopia, astigmatism, binocular vision disorders and/or visual fatigue syndrome) comprising: a base power profile; and at least one pulse (e.g., a plurality of pulses) selected to modify the base power profile and to form one or more on-axis focal points in front of, on, and/or behind a retinal image plane; wherein the ophthalmic lens is configured to provide a depth of focus that provides continuous (e.g., substantially continuous) vision from an object in distance to an object in the near vergences while focusing on a distance object.
G2. The ophthalmic lens of any of the G examples, wherein the depth of focus may provide a partial depth of focus at near objects and/or distance object and/or intermediate objects.
G3. The ophthalmic lens of any of the G examples, wherein the at least one pulse may be configured to create a diffraction effect to produce an image inside the human eye.
G4. The ophthalmic lens of any of the G examples, wherein the at least one pulse may be located on a front surface and/or a back surface of the ophthalmic lens.
G5. The ophthalmic lens of any of the G examples, wherein the at least one pulse may be formed in the matrix of the ophthalmic lens (e.g., between the front and back surfaces).
G6. The ophthalmic lens of any of the G examples, wherein the at least one pulse may be formed on a layer, a film, a lens precursor (e.g., a mold tool), a casting mold, a lens blank and/or formed on a fully finished lens as a post processing step.
G7. The ophthalmic lens of any of the G examples, wherein the at least one pulse may be formed by a material additive process (e.g., printing, stamping, dying, 3D printing, and/or a lithographic process).
G8. The ophthalmic lens of any of the G examples, wherein the at least one pulse may be formed by a material subtractive process (e.g., lasers, light energy, CO2 or UV, etching, thermal, and/or a chemical process).
G9. The ophthalmic lens of any of the G examples, wherein the at least one pulse may be formed by a material modification process (e.g., thermal, chemical changing the shape, refractive index and/or a chemical composition modification process).
G10. The ophthalmic lens of any of the G examples, wherein the at least one pulse may have a width of less than 0.3 mm.
G11. The ophthalmic lens of any of the G examples, wherein the at least one pulse may have a width of about 0.05 mm.
G12. The ophthalmic lens of any of the G examples, wherein the one or more pulses may comprise 1, 2, 3, 4, 5, 6, 7, 8, 9, 10, 11, 12, 13, 14, 15, 20, 25, 30, 35, 40, 45, 50, 60, 70, 80, 90, and/or 100 pulses.
G13. The ophthalmic lens of any of the G examples, wherein one of the one or more pulses may have a width of about 0.01 mm to 0.3 mm (e.g., about 0.01 mm, 0.02 mm, 0.03 mm, 0.04 mm, 0.05 mm, 0.1 mm, 0.15 mm, 0.2 mm, 0.25 mm, 0.3 mm, 0.01-0.03 mm, 0.01-0.05 mm, 0.02-0.05 mm, 0.05-0.1 mm, 0.1-0.2 mm, 0.2-0.25 mm, and/or 0.25-0.3 mm).
G14. The ophthalmic lens of any of the G examples, wherein the at least one pulse may have a height (e.g., a dioptric power) of between −100 D to +100 (e.g., +/−1 to 5 D, +/−1 to 10 D, +/−0.5 to 5 D, +/−0.5 to 10 D, +/−1 to 15 D, +/−1 to 20 D, +/−1 to 25 D, 1 to 30 D, +/−1 to 40 D, +/−1 to 50 D, +/−1 to 60 D, +/−1 to 70 D, +/−1 to 75 D, +/−1 to 80 D, +/−1 to 90 D, +/−1 to 100 D, +/−20 to 50 D, +/−25 to 50 D, +/−50 to 100 D, +/−75 to 100 D, +/−20 to 60 D, +/−25 to 75 D, and/or +/−30 to 50 D).
G15. The ophthalmic lens of any of the G examples, wherein the at least one pulse may have a shape selected from any combination of one or more of a square, conjoined of different features, triangles, rounded top, curved tops, slanted, oblique sides, and/or transition on the sides.
G16. The ophthalmic lens of any of the G examples, wherein the sides of a pulse may be parallel to each other (e.g., substantially parallel) or not parallel to each other.
G17. The ophthalmic lens of any of the G examples, wherein the tops of the pulses may be at least one of a planar, not planar, curved, monotonic, non-monotonic, periodic, and/or aperiodic profile (e.g., power profile).
G18. The ophthalmic lens of any of the G examples, wherein the at least one pulse may be a rapid, narrowband change in power.
G19. The ophthalmic lens of any of the G examples, wherein the at least one pulse may comprise a change in power comprising an increasing power component, a peak power component, and a decreasing power component relative to the base power profile.
G20. The ophthalmic lens of any of the G examples, wherein the sides of one of the at least one pulse may be connected to the base surface and a peak (or top) of the pulse may not be connected to any other feature on the lens.
G21. The ophthalmic lens of any of the G examples, wherein a first derivative of the pulse optical/power profile may be discontinuous at a boundary between the base power profile and the at least one pulse.
G22. The ophthalmic lens of any of the G examples, wherein the at least one pulse may include a discontinuity in the first derivative of the power profile in a least one location of the at least one pulse.
G23. The ophthalmic lens of any of the G examples, wherein the pulse may be a narrow width feature having a predefined width, power and/or position selected to change (e.g., reduce) at least one of the variability and magnitude of an RIQ associated with the base power profile.
G24. The ophthalmic lens of any of the G examples, wherein the change in RIQ may be at any portion of the RIQ plot.
G25. The ophthalmic lens of any of the G examples, wherein the change in RIQ may be at a particular vergence.
G26. The ophthalmic lens of any of the G examples, wherein one of the at least one pulses may be configured to reduce the peak RIQ (or the RIQ at any vergence) by 5% or less or 10% or less or 15% or less or 20% or less or 25% or less or 30% or less or 35% or less or 40% or less or 45% or less or 50% or less or 55% or less or 60% or less or 65% or less or 70% or less or 75% or less or 80% or less or 85% or less or 90% or less or 95% or less or between 5%-10% or between 5%-15%, or 10%-15% or 10% to 20% or 15%-25% or 20% to 25% or 25% to 50% or 30% to 50% or 50% to 60% or 50% to 75% or 60% to 75% or 75% to 95%
G27. The ophthalmic lens of any of the G examples, wherein the at least one pulse may be a positive powered pulse relative to the base power (e.g., the base power of the lens at the start of the pulse).
G28. The ophthalmic lens of any of the G examples, wherein the at least one pulse may be a negative powered pulse relative to the base power (e.g., the base power of the lens at the start of the pulse).
G29. The ophthalmic lens of any of the G examples, wherein the at least one pulse (e.g., a relatively positive, and/or negative pulse) may be configured to influence an extended depth of focus (EDOF).
G30. The ophthalmic lens of any of the G examples, wherein a relatively positive powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
G31. The ophthalmic lens of any of the G examples, wherein a relatively negative powered pulse may be configured to influence (e.g., change) the RIQ at any vergence depending on its height, width, shape and/or location (e.g., a positive pulse may influence the RIQ over a distance vergence or an intermediate vergence or a near vision vergence or an EDOF vergence range or beyond the visually useful range of vergences from far to near).
G32. The ophthalmic lens of any of the G examples, wherein the at least one pulse may be a relatively positive powered pulse and a relatively negative powered pulse.
G33. The ophthalmic lens of any of the G examples, wherein the ophthalmic lens may comprise a substantially equal number (e.g., 1, 2, 3, 4, 5, 6, 7, 8, 9, or 10) of relatively positive powered pulses and a relatively negative powered pulses.
G34. The ophthalmic lens of any of the G examples, wherein the one or more pulses may have a power to correct or contribute to one or more of a distance, intermediate and/or a near power (e.g., the one or more pulses may provide a depth of focus).
G35. The ophthalmic lens of any of the G examples, wherein the at least one pulse may have a power configured for the treatment of at least one refractive error (e.g., a myopic defocus and/or a hyperopic defocus) and/or may contribute an optical signal or retinal image quality associated with a vision correction or vision treatment or ocular condition.
G36. The ophthalmic lens of any of the G examples, wherein the one or more pulses may not correspond to one or more of a distance, intermediate and/or near power.
G37. The ophthalmic lens of any of the G examples, wherein the one or more pulses may be conjoined pulses.
G38. The ophthalmic lens of any of the G examples, wherein the one or more pulses may be a pair of conjoined pulses configured to form a ring, a portion or ring, or a plurality of portions of a ring.
G39. The ophthalmic lens of any of the G examples, wherein the one or more pulses may be a plurality of pairs of conjoined pulses configured to form a plurality of rings.
G40. The ophthalmic lens of any of the G examples, wherein the at least one pulse may be configured to modify (e.g., increase or decrease) the RIQ for myopia control.
G41. The ophthalmic lens of any of the G examples, wherein the at least one pulse may be configured to modify (e.g., increase and/or decrease) the peak RIQ to below 0.5 (e.g., below 0.5, 0.45, 0.4, 0.35, 0.3, 0.25, 0.2, 0.15, 0.1, 0.05, 0).
G42. The ophthalmic lens of any of the G examples, wherein the at least one pulse may include a pulse located in the center of the lens.
G43. The ophthalmic lens of any of the G examples, wherein the at least one pulse may be created on a portion of the lens in a non-radial direction (e.g., to create an asymmetrical, non-annular, and/or non-concentric pulse).
G44. The ophthalmic lens of any of the G examples, wherein the at least one pulse may vary across a particular dimension of the pulse (e.g., any combination of one or more of a width, height, and/or shape varies in a particular dimension and/or direction).
G45. The ophthalmic lens of any of the G examples, wherein the shape of the pulse may include parallel or non-parallel side and a planar or non-planar top.
G46. The ophthalmic lens of any of the G examples, wherein the ophthalmic lens may be manufactured using at least one of lathing, molding, spinning, printing, etching, sputtering
G47. The ophthalmic lens of any of the G examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D, ±3D, ±3.1D, ±3.2D, and/or ±3.25D)), and wherein (1) the maximum RIQ value of the independent peaks is between about 0.11 (e.g., 0.09, 0.1, 0.11, 0.12, 0.13, 0.14 or 0.15) and about 0.45 (e.g., 0.42, 0.43, 0.44, 0.45, 0.46, 0.47 or 0.48) or (2) the maximum RIQ value of the independent peaks is less than about 0.45 (e.g., 0.42, 0.43, 0.44, 0.45, 0.46, 0.47 or 0.48).
G48. The ophthalmic lens of any of the G examples, wherein the ophthalmic lens may provide a through focus retinal image quality (RIQ) with one or more (e.g., 1, 2, 3, 4, or 5) independent peaks (e.g., over a vergence range of about ±3D (e.g., ±2.75D, ±2.8D, ±2.9D ±3D, ±3.1D ±3.2D, and/or ±3.25D)), and wherein an RIQ area of the one or more independent peaks is about 0.16 Units*Diopters (e.g., 0.13, 0.14, 0.15, 0.16, 0.17, 0.18 or 0.19) or less.
G49. The ophthalmic lens of any of the G examples, wherein the ophthalmic lens may provide a through focus retinal image quality (RIQ) with one or more (e.g., 1, 2, 3, 4, 5, 6, 7, 8, 9, 10, 11, 12, 13, 14, 15, 16, 17, 18, 19, 20, 21, 2, 23, 24, and/or 25) independent peaks (e.g., over a vergence range of about ±3.0D (e.g., ±2.75D, ±2.8D, ±2.9D, ±3D, ±3.1D, ±3.2D, and/or ±3.25D)), and wherein there is at least one or more independent peaks (e.g., 1, 2, 3, 4, 5, 6, 7, 8, 9, 10, 11, 12, 13, 14, 15, 16, 17, 18, 19, 20, 21, 2, 23, 24, and/or 25 peaks).
G50. The ophthalmic lens of any of the G examples, wherein the at least one pulse may be about 20-400 μm wide (e.g., about 15 μm, 20 μm, 30 μm, 40 μm, 50 μm, 60 μm, 70 μm, 75 μm, 80 μm, 90 μm, 100 μm, 110 μm, 120 μm, 125 μm, 130 μm, 140 μm, 150 μm, 160 μm, 170 μm, 175 μm, 180 μm, 190 μm, 200 μm, 210 μm, 220 μm, 225 μm, 250 μm, 275 μm, 300 μm, 325 μm, 350 μm, 375 μm, 400 μm, and/or 425 μm wide).
G51. The ophthalmic lens of any of the G examples, wherein the at least one pulse may comprise a plurality of narrow and/or annular concentric optical zones located on at least one of the front surface and/or the back surface and/or within the matrix of the ophthalmic lens and formed by line curvatures.
G52. The ophthalmic lens of any of the G examples, wherein the ophthalmic lens may be one of a contact lens, an intraocular lens, and/or a spectacle lens.

It will be understood that the embodiments disclosed and defined in this specification extends to all alternative combinations of two or more of the individual features mentioned or evident from the text or drawings. All of these different combinations constitute various alternative aspects of the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1-16. (canceled)

17. An ophthalmic lens configured to correct and/or treat at least one condition of the eye comprising: a base power profile; and at least one pulse selected to modify the base power profile and to form one or more on-axis focal points in front of, on, and/or behind a retinal image plane; wherein the ophthalmic lens is configured to provide a depth of focus that provides continuous or substantially continuous vision from an object in distance to an object in the near vergences while focusing on a distance object.

18. (canceled)

19. The ophthalmic lens of claim 17, wherein the at least one pulse is configured to create a diffraction effect to produce an image inside the human eye.

20-25. (canceled)

26. The ophthalmic lens of claim 17, wherein the at least one pulse has a width of less than 0.3 mm.

27-28. (canceled)

29. The ophthalmic lens of claim 17, wherein one of the one or more pulses has a width of about 0.01 mm to 0.3 mm.

30. The ophthalmic lens of claim 17, wherein the at least one pulse has a dioptric power of between −100 D to +100.

31-33. (canceled)

34. The ophthalmic lens of claim 17, wherein the at least one pulse is a rapid, narrowband change in power.

35. The ophthalmic lens of claim 17, wherein the at least one pulse comprises a change in power comprising an increasing power component, a peak power component, and a decreasing power component relative to the base power profile.

36. (canceled)

37. The ophthalmic lens of claim 17, wherein a first derivative of the pulse optical/power profile is discontinuous at a boundary between the base power profile and the at least one pulse.

38. The ophthalmic lens of claim 17, wherein the at least one pulse may includes a discontinuity in the first derivative of the power profile in a least one location of the at least one pulse.

39-42. (canceled)

43. The ophthalmic lens of claim 17, wherein the at least one pulse is a positive powered pulse relative to the base power.

44. The ophthalmic lens of claim 17, wherein the at least one pulse is a negative powered pulse relative to the base power.

45-47. (canceled)

48. The ophthalmic lens of claim 17, wherein the at least one pulse is a relatively positive powered pulse and a relatively negative powered pulse.

49. The ophthalmic lens of claim 17, wherein the ophthalmic lens comprises a substantially equal number of relatively positive powered pulses and a relatively negative powered pulses.

50-52. (canceled)

53. The ophthalmic lens of claim 17, wherein the one or more pulses are conjoined pulses.

54-56. (canceled)

57. The ophthalmic lens of claim 17, wherein the at least one pulse is configured to a peak RIQ of the ophthalmic lens to below 0.5.

58. The ophthalmic lens of claim 17, wherein the at least one pulse includes a pulse located in the center of the lens.

59-66. (canceled)

67. The ophthalmic lens of claim 17, wherein the at least one pulse comprises a plurality of narrow and/or annular concentric optical zones located on at least one of the front surface and/or the back surface and/or within the matrix of the ophthalmic lens and formed by line curvatures.

68. (canceled)
